(19) United States
(12) Patent Application Publication
(10) Pub. No.: US 2007/0185390 A1
(43) Pub. Date: Aug. 9, 2007

(54) Information workflow for a medical diagnostic workstation

(75) Inventors: David G. Perkins, Tully, NY (US); Douglas J. Linquest, West Linn, OR (US); Kristin A. Metz, Camillus, NY (US)

Correspondence Address: MARJAMA & BILINSKI LLP, 250 SOUTH CLINTON STREET, SUITE 300, SYRACUSE, NY, 13202, US

(73) Assignee: Welch Allyn, Inc., Skaneateles Falls, NY (US)

(21) Appl. No.: 11/703,470

(22) Filed: Feb. 7, 2007

Related U.S. Application Data
(63) Continuation-in-part of application No. 11/131,015, filed on May 17, 2005.
Application No. 11/131,015 is a continuation-in-part of application No. 10/643,487, filed on Aug. 19, 2003.
(60) Provisional application No. 60/771,502, filed on Feb. 8, 2006.

(51) Int. Cl. A61B 5/00; G06Q 10/00

(52) U.S. Cl. 600/300; 600/301; 705/2

(57) ABSTRACT

An information workflow for a medical diagnostic workstation in which patient data is captured, arranged and displayed in predetermined formats for a user in the handling of patients. The workflow permits vitals capture and storage and creation of a comprehensive patient record in which the workstation can operate in a stand-alone or network connected mode.

### CROSS REFERENCE TO RELATED APPLICATIONS

This patent application is a nonprovisional application claiming priority under 35 U.S.C. 119 based upon a provisional application, U.S. Ser. No. 60/771,502, which was filed on Feb. 8, 2006. This patent application also claims priority under 35 U.S.C. 120 as a continuation in part (CIP) application of U.S. Ser. No. 11/131,015, filed on May 17, 2005, which is a continuation in part (CIP) application of U.S. Ser. No. 10/643,487, filed Aug. 19, 2003, the entire contents of each herein being incorporated by reference.

### FIELD OF THE INVENTION

This invention relates to the field of diagnostic medicine and in particular to an information workflow for a medical workstation in order to create and maintain fully comprehensive patient records.

### BACKGROUND OF THE INVENTION

The staff of a medical/surgical floor of a typical hospital or other health care facility is under increasing amounts of pressure. Contributing to this pressure is the pervasive nursing shortage that has translated into a lower nurse to patient ratio. Therefore, the staff works longer hours and increased overtime. As a result of the above-noted shortage, it has been noted that lapses in transcribing patient readings during rounds and other associated errors have increased. Formerly, patient vital signs data were taken by a registered nurse (RN), but now these readings are often taken numerous times (as many as six or more readings) per day by nursing aides (also referred to as Patient Care Technicians (PCTs)), who must cover more patients and often have no or little clinical training. In addition and in an effort to ease the above staffing strains, many hospitals utilize more temporary contract or “traveler” nurses who float between sites. As a result, users of patient monitoring equipment are transitory and must learn new internal procedures very quickly, exacerbating the above problems. The time consumption required in the recording of patient measurements and in attempting to maintain an efficient and comprehensive patient record(s) among multiple caregivers adds to the above-noted problems.

Currently, PCTs often use a cart having a number of patient diagnostic devices that can include various automated and/or manual blood pressure, thermometry, and pulse oximetry apparatus used to take patient vital signs over the course of a typical hospital stay. As noted, a PCT may likely take six readings (or more) per day over an average hospital stay of about five days. Typically, the above-noted diagnostic devices are not integrated together on the cart, but rather are arranged in a piecemeal fashion thereupon. Though integrated vital sign monitoring (VSM) devices, such as those sold and manufactured by Welch Allyn, Inc., of Skaneateles Falls, New York, are commonly known in the field, no such monitoring device is presently used in a manner that maximizes its effectivity, for example, on a clinical workstation.

Vital sign readings, when taken, using the above apparatus, are often written onto a loose worksheet or often onto scraps of paper. At the end of rounds, these readings are then copied by hand onto the patient's chart on a “vitals” sheet. If anomalous readings are detected, the RN or attending physician is notified. Otherwise, the RN or physician is usually not consulted and often will not or may not get the opportunity to review any of the readings concerning a particular patient(s).

Upon examination and if any vital signs readings are suspect in any way, the RN will often send the PCT to capture additional patient readings. In the meantime, even if a significant change in the patient's vitals has been detected, additional time has been inefficiently consumed and is therefore lost. In addition, there is other patient-relevant data that may not be obtained directly from a diagnostic device, such as the position of the patient during testing, the position of the monitoring sensor(s) on the patient, the comfort level of the patient and other data is often not readily captured during rounds and often is entered (if such data is entered at all) based upon the best recollection of the caregiver, making the reliability of such data uncertain. It is further possible that in the current manner of testing described above, many vital signs variations are not caught or otherwise detected or noted until the patient's condition has significantly changed.

Though the problems are arguably less involved, there are similar generalized needs in other clinical settings, such as physician's offices, in order to be able to more efficiently and accurately conduct and document patient clinical encounters.

Numerous patient diagnostic workstations are known in the current art, such as described by U.S. Pat. No. 5,687,717 to Halpern et al. These workstations employ a plurality of diagnostic and therapy modules that are arranged within drawers in a wheeled assembly. However, this system does not provide a convenient means for importing data from other systems or devices or for capturing and maintaining patient records, outside of certain physiologic readings.

### SUMMARY OF THE INVENTION

According to one aspect, there is provided an electronic data management system that comprises at least one computing device, at least one display, at least one input device and at least one vital signs collecting device, wherein said each of said at least one computing device, and said at least one display are interconnected to one another in a diagnostic workstation and said at least one vital signs collecting device is at least one of directly integrated into said workstation or is configured for connection therewith, but is not a structural part of said workstation. The at least one computing device is programmed to receive and manage vital signs readings relating to at least one patient and the system further includes a plurality of selectively accessible display screens for creating an electronic patient record and managing data relating to the at least one patient.

According to one version, data from the at least one device that is not structurally connected can be selectively imported into the workstation. This device can be, for example, wirelessly connected to the workstation. According to another aspect, vital data can be captured, as well as qualifier data that is not directly captured by a collecting device wherein the data can be selectively modified and stored. According to yet another aspect, various patient specific data can be selectively accessed in addition to tabular and/or graphical data that is stored by the workstation for display.

The workstation can be operated to capture information using at least one resident vitals collecting device or readings from remote devices can be imported, with patient context, either within a network or as stand-alone. The information or data can be presented so as to provide notification to a user when out of range conditions are present, either in terms of equipment malfunction, as a result of inputting errors or due to changes in patient condition.

Notifications or alerts can be programmed by the workstation for certain vital sign parameters based on a patient by patient basis, by a group, such as a hospital floor, or based upon a default or preselected range of alarm limits.

The workstation further provides means for enabling literally all aspects of a patient's status and condition to be tracked irrespective of the user of the workstation through various use of reminder windows, patient notes windows and message windows that enable communication between caregivers. For example, a caregiver who is late entering a shift or a traveling nurse or clinician through use of the preceding note and message features is kept current on each of the patients they are handling. These tools simplify communication between users who, while covering the same patients, may not have adequate time or occasion to otherwise interact.

In addition, the workstation further provides the ability for the user to sign off or approve on any captured vitals or other data before entering them into storage, enabling the opportunity to either verify or complete the necessary readings for a patient. Nurses may also be permitted to sign off upon readings that are taken under their guidance and medical license by other non-licensed caregivers.

The workstation is connectable to various information systems so as to provide complete and updated electronic patient records, for example, into a hospital information system having a database.

According to another aspect, there is provided an electronic patient data management system that includes a computing device, at least one display, and at least one vital signs collecting device, each of said computing device, said at least one display and said at least vital signs collecting device being interconnected to one another in a diagnostic workstation. Data relating to at least one patient is captured and managed using a plurality of selectively accessible electronic display screens.

An advantage of the herein described workstation is that a plurality of patients can effectively and reliably handled in a hospital facility using at least one workstation to maintain all measured data pertaining to the patients during a hospital stay.

A further advantage is that the herein described workstation permits vital sign data to be captured using a plurality of devices, either structurally part of or connected to the workstation.

These and other aspects, features and advantages will be readily apparent from the following Detailed Description which should be read in conjunction with the accompanying drawings.

### BRIEF DESCRIPTION OF THE DRAWINGS

FIG. 1 is a front view of an exemplary medical diagnostic workstation that is made in accordance with the present invention;

FIGS. 2(a) and 2(b) represent various side perspective views of the medical diagnostic workstation of FIG. 1;

FIG. 3 is a rear perspective view of the medical diagnostic workstation of FIGS. 1 and 2;

FIG. 4 is an enlarged view of a portion of the medical diagnostic workstation of FIG. 3;

FIG. 5 is a generalized functional schematic block diagram of the medical diagnostic workstation of FIGS. 1-4;

FIG. 6 is a system diagram of a hospital computer network, including at least one medical diagnostic workstation according to FIGS. 1-4;

FIG. 7 is an exemplary log-in display screen for the medical diagnostic workstation;

FIG. 8 is an exemplary user display screen of the medical diagnostic workstation of FIG. 5;

FIGS. 9(a) and 9(b) depict alternative methods provided by the diagnostic workstation to search patients handled by the workstation;

FIG. 10 is the exemplary user display screens of FIG. 8, including a pop-up locations window;

FIG. 11 depicts the exemplary user's display screen of FIGS. 8 and 10 including an exemplary patient reveal window relating to an identified patient;

FIGS. 12 and 13 is a set reminders display window relating to the identified patient of FIG. 1;

FIG. 13 is an exemplary reminders display window;

FIG. 14 is an exemplary reminders reveal window that can be selectively displayed by the workstation;

FIG. 15A is a sample message window;

FIG. 15B is a sample exemplary incoming message that can be displayed by the medical diagnostic workstation;

FIG. 15C is a sample messages list window;

FIG. 16 is an exemplary vitals capture display screen of the workstation;

FIGS. 17-20 represent various aspects of the vitals capture display screen of FIG. 16;

FIG. 21 is a confirmation of vitals capture display screen displayed by the workstation relating to the data captured in FIGS. 16-20, prior to storage by the medical diagnostic workstation;

FIGS. 22-24 are other exemplary examples of the vital capture display screen of FIG. 16;

FIG. 25A is an exemplary set notifications/alerts display screen of the diagnostic workstation;

FIG. 25B is another exemplary alert set-up display screen of the diagnostic workstation;

FIG. 25C is a version of the alerts set-up display screen of FIG. 25B for a specific location or group;

FIGS. 26 and 27 are exemplary patient summary display screens of the workstation;

FIGS. 28A and 28B depict other versions of exemplary patient summary display screens of the workstation according to another aspect;

FIG. 29A is an allergies and sensitivities display window;

FIG. 29B depicts a pop-up window for the display window of FIG. 29A;

FIG. 29C is an edit allergies window;

FIG. 29D is an allergies window;

FIG. 29E is a set allergies window;

FIG. 30 depicts the patient summary display screen of FIGS. 26 and 27 and further displaying a vitals notification window relating to a particular portion of an identified patient's record;

FIG. 31 depicts an exemplary patient summary display screen according to yet another aspect;

FIGS. 32-34 depict various display screens for importing vital sign data from a separate remote medical device to the workstation; and

FIGS. 35 and 36 depict other exemplary patient reveal windows similar to those depicted according to FIG. 11 for other identified patients from the list of patients in the user's display screen of FIGS. 8 and 10.

### DETAILED DESCRIPTION

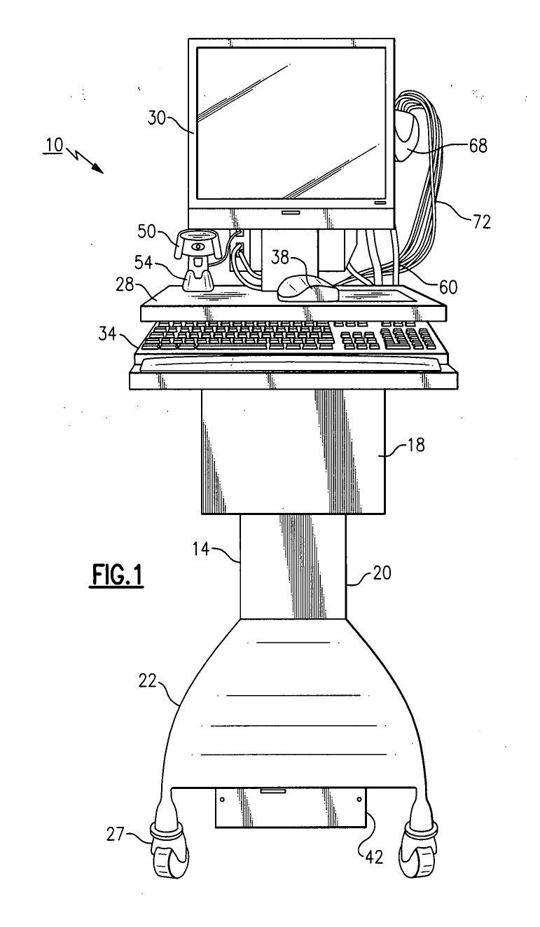

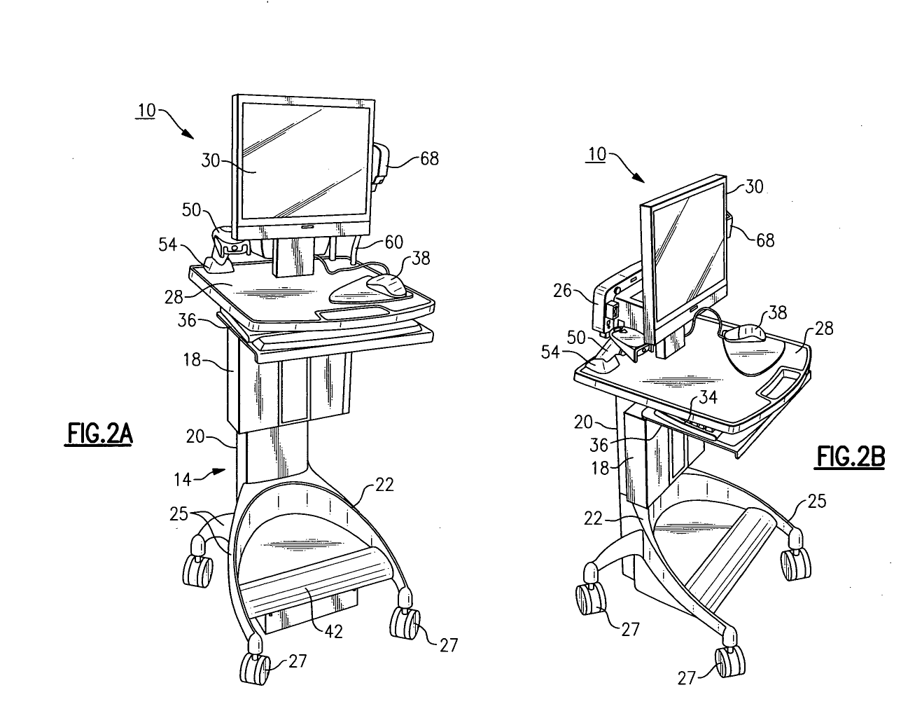

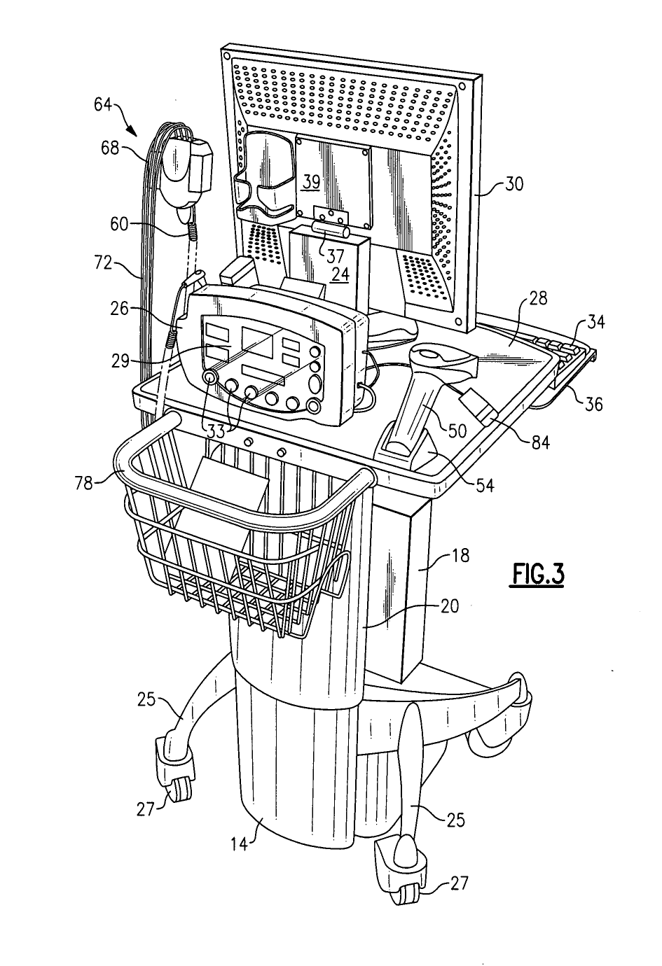

Referring to FIGS. 1-4, there is shown a medical diagnostic workstation 10 made in accordance with an exemplary embodiment. Prior to a detailed discussion, it should be noted that it will be readily apparent to those of sufficient skill that there are numerous and varied modifications that are possible within the intended ambit and scope of the inventive concepts presented herein and for that reason, all possible variations cannot possibly be described in detail. In addition, certain terminology is used frequently herein in order to provide a suitable frame of reference with regard to the accompanying drawings. This terminology is not intended to be limiting, unless specifically indicated otherwise.

The medical diagnostic workstation 10, according to this exemplary embodiment, is defined by a wheeled chassis 14 that is configured to support a plurality of components, as described below. The wheeled chassis 14 of the herein described workstation 10 is further defined by a base portion 22 that retains the vertically extending post member 20, the base portion further including a set of spaced legs 25 having individual casters 27 attached at the ends of each of the legs. According to this embodiment, a computing device 18 is attached to the exterior of a vertically extending post member 20. More specifically, the computing device 18 can alternatively include at least one of a laptop computer, a personal computer (PC), a tablet PC and a portable data terminal (PDT).

A resident vital signs collecting device 26 capable of measuring specific physiologic parameters of a patient, including, for example, heart rate, body temperature, pulse oximetry, and blood pressure, is attached to an upper portion 24 of the vertically extending post member 20 of the diagnostic workstation 10. The vital signs collecting device 26, according to this exemplary embodiment, further includes a display 29 as well as a user interface that includes a series of actuable buttons 33 used to manually control the operation of the collecting device 26. A display 30 is oppositely mounted relative to the upper portion 24 of the vertically extending post member 20, the display being pivotably mounted by a bracket 37 to an attachment plate 39 that is secured to the rear side of the display 30. According to this embodiment, the resident vital signs collecting device 26 is a Vital Sign Monitor 300 Series, manufactured and sold by Welch Allyn, Inc., though the specifics of the device can be suitably varied, as described herein. That is to say, other vital signs collecting devices can be alternately provided. In addition and as described in greater detail herein, other medical devices can be connected directly to or in relation to the herein described workstation 10 such as, for example, a Welch Allyn Vital Sign Monitor (VSM) 52000 series, a Spot Vital Signs device or a Spot Vital Signs Lxi device.

Still referring to FIGS. 1-4, the medical diagnostic workstation 10 includes a keyboard 34 that is fitted within a sliding tray drawer 36, which is disposed beneath the horizontal work surface 28. The keyboard 34 is connected to the computing device 18 and display 30, though other input devices such as a trackball (not shown), mouse 38, wireless stylus or other suitable device can be further or alternatively utilized.

The medical diagnostic workstation 10 further includes a presentation bar code scanner 50, such as, for example, the Image Team 4620 Cordless 2D Imager Device, manufactured by Hand Held Products of Skaneateles Falls, New York, the scanner being retained within a retaining cradle 54 provided on the top of the horizontal work surface 28. The herein described scanner 50 comprises a pistol-grip shaped housing that retains at least one electronic imaging device and an illumination source such that machine-readable information (i.e., 1-D and 2-D bar code symbologies) can be scanned and interpreted (decoded) by this apparatus, which is interconnected to the computing device 18 of the workstation 10. It should be noted that in lieu of the specific scanner shown, other forms of scanning devices, for example RFID interrogating devices and other apparatus that are capable of reading other forms of machine readable information, such as, for example, those equipped for OCR (Optical Character Recognition) can also be used herewith. The retaining cradle 54, according to this embodiment, is situated on the horizontal work surface 28 in order to provide ample room to permit items to be brought to or “presented” to the scanner 50; for example, medications or other items such as cuffs, syringes and the like that could be used by a caregiver/patient during the course of a typical hospital stay. The scanner 50 can also be operated when removed from the retaining cradle 54, such as, for example, to read a patient's wristband or physician's badge for identification thereof, as described in greater detail below.

Oppositely situated on the horizontal work surface 28 relative to the vertically extending post member 20 and the presentation scanner 50, a vertically extending flexible goose-neck 60 is attached at one end, the depending end of the gooseneck receiving an ECG electrode assembly 64. The ECG electrode assembly 64 includes a housing or harness 68, as well as a set of tethered leadwires/electrodes 72 that are configured for attachment to a patient (not shown) in a conventional manner so as to receive ECG data therefrom. The analog ECG signals received from the electrodes 72 are processed and converted into digital signals comprising appropriate waveforms (i.e., vectors) depending on the number of leads (e.g., 12 lead, 15 lead) that are attached in a conventional and known manner to the patient. This input can be directed to the computing device 18 or to the resident vital signs collecting device 26, in the instance that the vital signs collecting device is configured to accept ECG data.

The diagnostic workstation 10 described herein is intended to be moved between a plurality of patient rooms and to that end includes a portable power source 42, such as at least one NiMH or other portable rechargeable battery, disposed herein at the base portion 22 of the wheeled chassis 14, thereby enabling the resident vital signs collecting device 26, the computing device 18, the display 30 and the presentation scanner 50. It should be noted that each of the foregoing devices can also include separate batteries (not shown) that can be recharged while each device is attached to the diagnostic workstation 10.

The workstation 10, according to this exemplary embodiment, further permits loose or unattached items to be stored, for example, in a receptacle 78 provided along the rear side thereof. The receptacle 78 is mounted by fasteners or other suitable means to the wheeled chassis 14 and more specifically to the rear facing side of the horizontal work surface 28. Alternatively, the receptacle can be integrally provided. The receptacle 78 further includes a handle 80 to enable the workstation 10 to be easily pushed or pulled between stations in a hospital or similar environment, such as between a plurality of patient locations. The design of this particular workstation can include additional receptacles, as needed, such as shown and described in the above cross-referenced Ser. Nos. 11/131,015 and 10/643,817 applications. In addition, the horizontal work surface 28 of the workstation 10 can also be selectively raised and lowered along the vertically extending post member 20 by means of a spring or gas-assisted lever (not shown), also as described in the above cross referenced applications, allowing the work surface 28 and the attached components to be suitably positioned, depending, for example, on the caregiver/user.

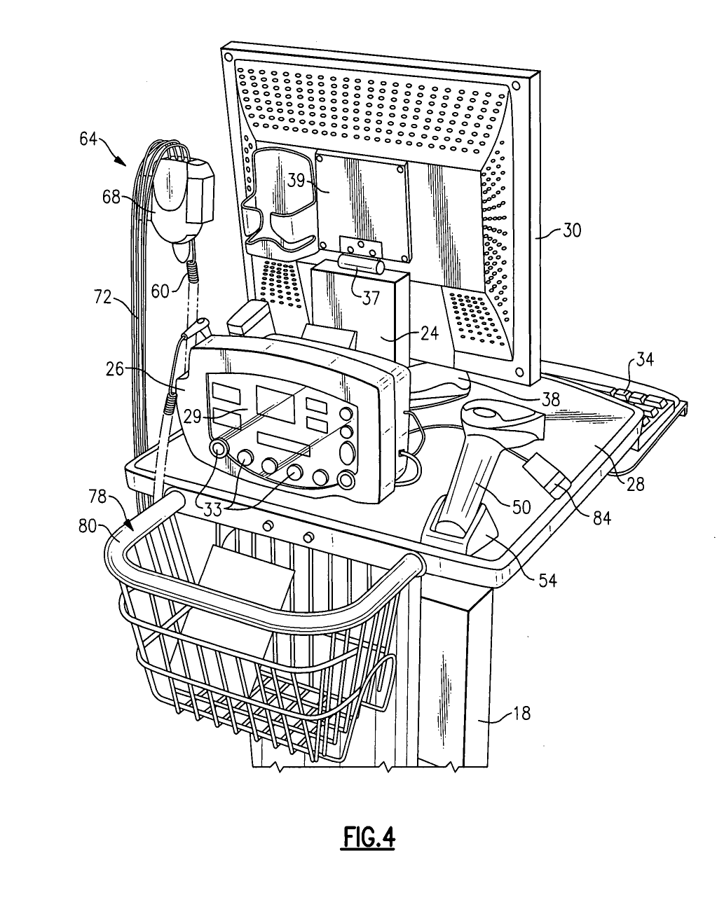

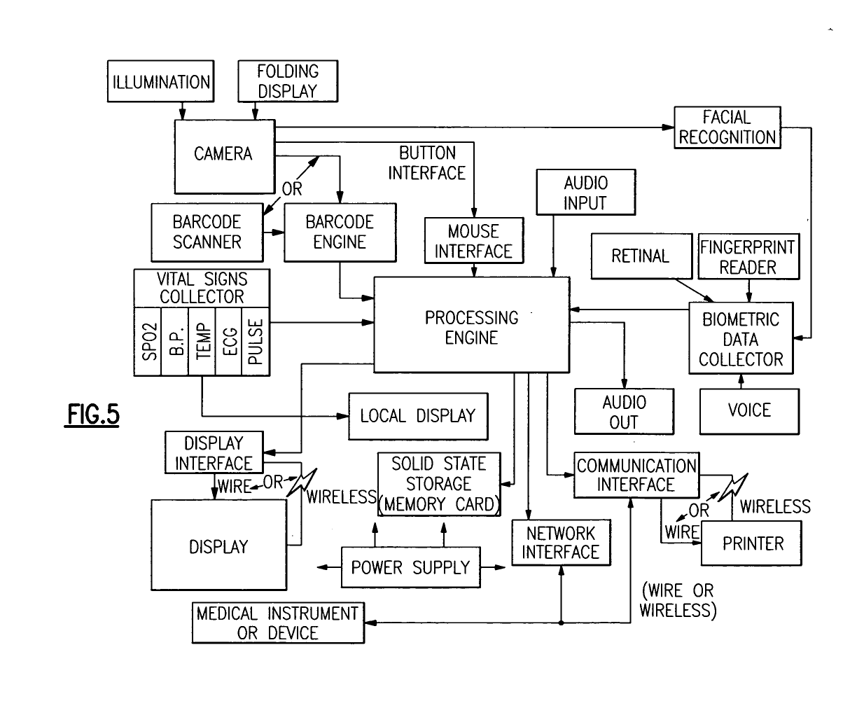

As previously noted, the resident vital signs collecting device 26 used according to this embodiment is a Welch Allyn VSM 300 Series device that includes a plurality of physiological parameter measuring modules that are integrated into a common housing. These modules include a temperature measuring module, a pulse oximetry module and a blood pressure measuring module. Each of these modules include physiologic sensors that are attached to a patient (not shown) and have connecting ends that are received by mating ports provided on the exterior of a device housing. Such connection is commonly known to those of skill in the field and requires no further discussion. By way of example, a finger pulse-oximeter sensor 84 of the vital signs collecting device 26 is illustrated in FIGS. 3 and 4.

Referring to FIG. 5, there is illustrated a schematic block diagram of the functional components of the exemplary medical diagnostic workstation 10 wherein the computing device 18 includes a processor or processing engine that interfaces with the vital signs collecting device 26, as well as the bar code scanner 50 and other apparatus, including remote devices that are resident to the workstation 10. Additional details relating to this diagram are provided in U.S. Ser. No. 10/643,487, the entire contents of which have been previously incorporated by reference in their entirety. In brief, the workstation 10 includes the above noted components and can communicate with remote devices, either directly or through a network interface, such that additional data relating to a patient can be received, collected, stored and transferred, as needed.

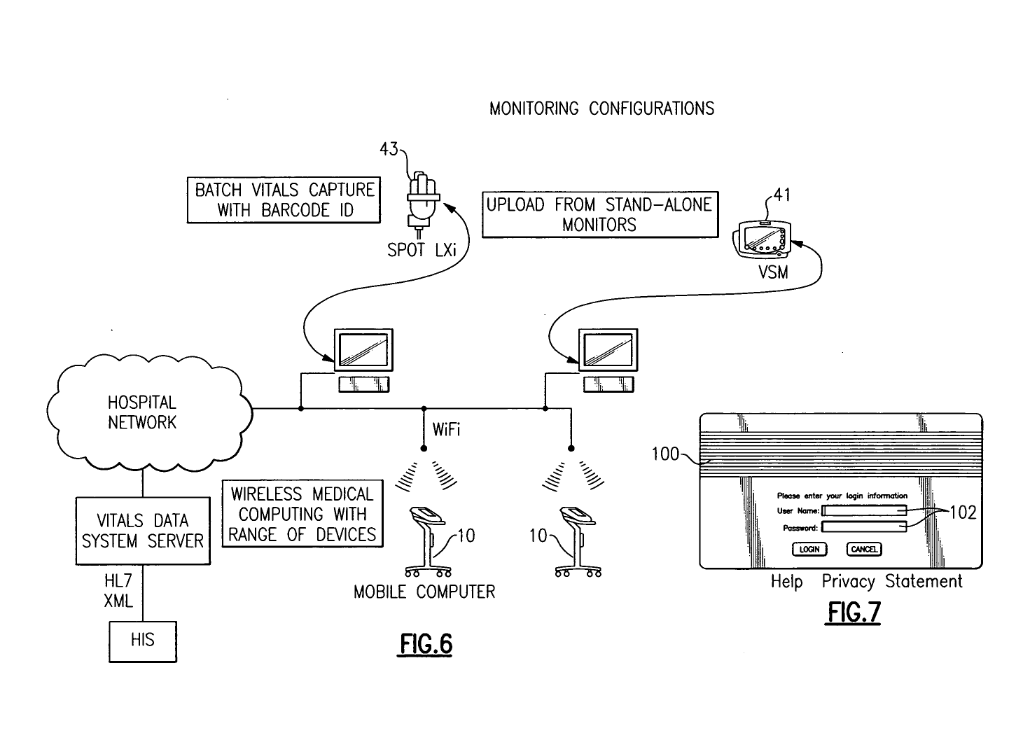

A typical network architecture is herein described with reference to FIG. 6 that involves at least one medical diagnostic workstation 10 as herein described in conjunction with either stand-alone or vital sign devices 41, 43 that are already incorporated onto the workstation itself or are otherwise located in the hospital or health care facility, respectively. In accordance, the diagnostic workstation 10 can be brought into a patient's area and be directly connected to a stand-alone patient monitoring or other diagnostic instrument or device 41. Readings can then be imported for storage by the workstation 10, as described in greater detail below. Alternatively and/or in addition to the resident vital signs collecting device 26, FIG. 1, a separate vital signs collecting device 43 can be wirelessly connected to the workstation 10, using a Wireless Fidelity (WiFi) connection, such as an IEEE 802.11 (b) or (g) connection, or a similar wireless protocol, and can download the readings taken to the workstation for importation thereof. According to one version, the latter can be done using the Spot LXi vital signs monitor manufactured by Welch Allyn, Inc. This specific vital signs monitoring device includes its own resident bar-code scanner to better insure patient context. This device also includes sufficient memory to store a predetermined number (e.g. up to fifty) of patient readings along with user identifications. The device further includes a user interface enabling users to enter manually captured readings, such as pain levels or respiration. In the present embodiment, the herein described diagnostic workstation 10 can be used to capture vital signs from a group of patients during typical patient rounds. At the end of rounds, the workstation 10 is connectable, for example, to a hospital network computer to upload the readings, storing the readings in a database, confirming patient identifiers, and sending the readings to other HIS (Health Information System) systems that desire the data.

The software used in the computing device 18 of the herein described exemplary workstation 10 includes an Intel or other suitable processor that utilizes a Windows XP or other comparable operating system and preferably includes a Web browser. Typical requirements for a server used in accordance with the present workstation 10 include a Pentium IV x86 dual-core 3.4 GHz or better processor having additional processor speed, as required. The specific computing device 18 is equipped with a memory of approximately 4 GB or greater with an 18.2 GB Ultra 320 SCSI 15,000 rpm Hard Drive, wherein additional database storage may be recommended and a CD-ROM capable of reading CD-RWs (any speed). An Ethernet connection requires approximately 1 GB between servers. As far as the workstation 10 itself and according to this embodiment, the specific processor used is a 750 MHz Pentium (or greater) having 256 MB (or greater) in memory and 500 MB free (or greater) disk space. The display 30 has a resolution of 1024×768 with 24 bit color (or greater). As far as an Ethernet connection, a 100 Mbps LAN (Local Area Network) or better is recommended.

As now will be further detailed, the diagnostic workstation 10 is programmed to enable the navigation of various selectively accessible display screens for the capture of vital sign and other patient-related data and for the formation and maintenance of at least one patient record. The information workflow pertaining to the navigation of the display screens, the capture and importation of vital signs and other data and the formation of the patient record and reports is herein described in greater detail in accordance with a detailed examples.

The following discussion relates to the information workflow of the exemplary diagnostic workstation 10 and in particular to a set of selectively accessible display screens depicting the workflow. Turning first to FIG. 7 and upon startup of the medical workstation 10, a first display screen that is opened and viewed in the display 30 of the workstation 10 is a log-in display screen 100. The log-in display screen 100 includes a pair of data entry fields 102, wherein entry of an authorized user name and a password is required before log-in can successfully occur. Each of the foregoing data items are stored, either in the memory of the computing device 18 of the workstation 10 or can be accessed from the workstation over the hospital computer network, as shown in FIG. 6, for verification. An authorized user's name can be entered using the scanner 50 to scan the badge of a physician or nurse or the keyboard 34 or mouse 38 can be used to enter the user's name. The password can be entered using the keyboard 34, mouse 38, or other input device.

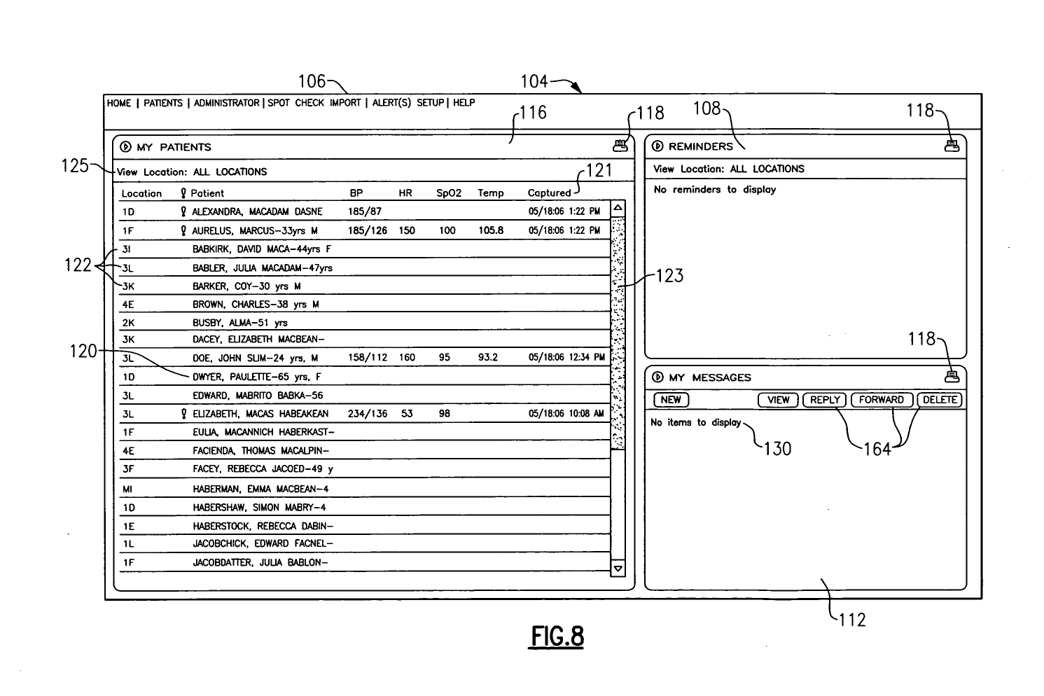

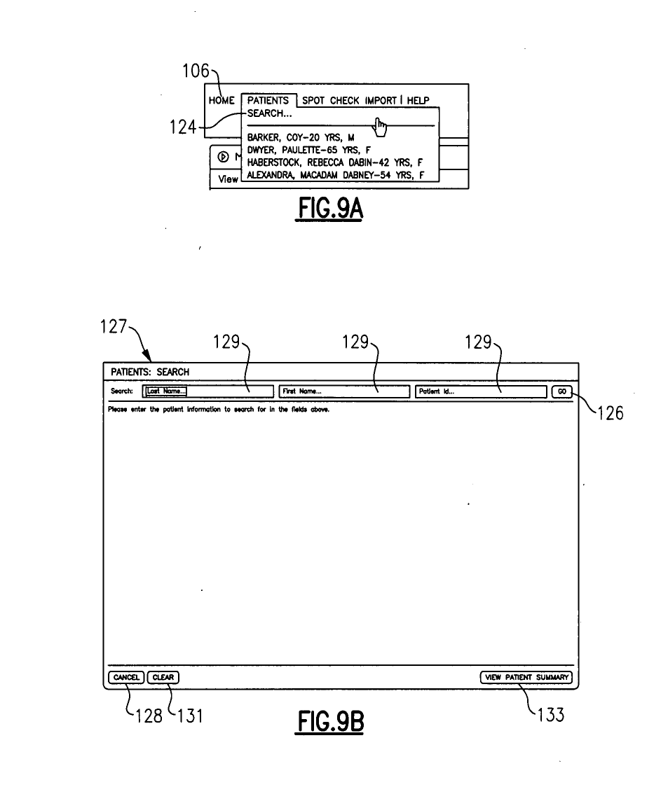

Upon successful login by an authorized user, a user's display screen 104 for an authorized user is accessed, as shown in FIG. 8, the user's display screen according to this embodiment including three (3) primary windows; namely, a Reminders window 108, an Inbox or Messages window 112 and a List Patients or Patients window 116, respectively. At the top of the user's display screen 104 is a global header 106 that includes a plurality of display options used in conjunction with the workstation 10. This header 106 is included at the top of several selectively accessible display screens, as discussed herein, and provides a means for navigating between various workstation functions. Each of the listed options in the global header 106 is discussed in greater detail below. The List Patients window 116, as shown in FIG. 8, includes a tabular list of patients 120 that are handled or assigned to the user of the diagnostic workstation 10, along with their most recently captured vitals data. Referring to FIGS. 9(a) and 9(b), the patients list 120 can be searched either alphabetically, forward or reverse, or by location as shown in FIG. 9(b). In addition, the format of the list 120 can also be modified, e.g., alphabetically, forward/reverse or by location. A vertical scroll bar 123 enables the user to advance through the tabular list 120, as needed. Clicking onto the patients option in the global header 106 enables the patients list to be searched, as shown in FIG. 9(a). Clicking on the search locator 124 in the window of FIG. 9(a) causes a patients search window 127 to be accessed for display, as shown in FIG. 9(b). The patients search window 127 includes a plurality of search data fields 129, enabling a user to search for a patient by entry of a sufficient number of characters of the patient's first name, last name and/or patient identification number, respectively. Search results are obtained by clicking on a go button 126 located on the top right hand corner of the patient search window 127.

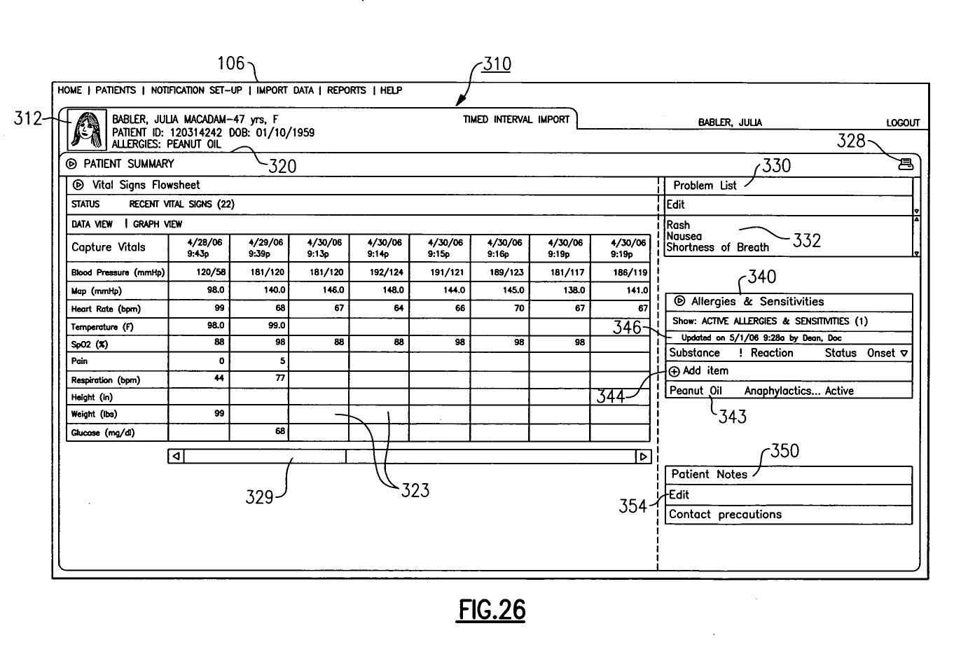

The patient search window 127 further includes a number of action or navigation buttons at the bottom of the search window including a cancel button 128, a clear button 131 and a view patient summary button 133, respectively. Clicking on the cancel button 128 returns the user to the user's display screen 104, FIG. 8. Clicking on the clear button 131 clears each of the search data fields 129 and the search results. Clicking on the view patient summary button 133 causes a patient summary display screen 310, FIG. 26, for the identified patient to be displayed by the workstation 10. This latter display screen and its functionality is described in greater detail in a succeeding section.

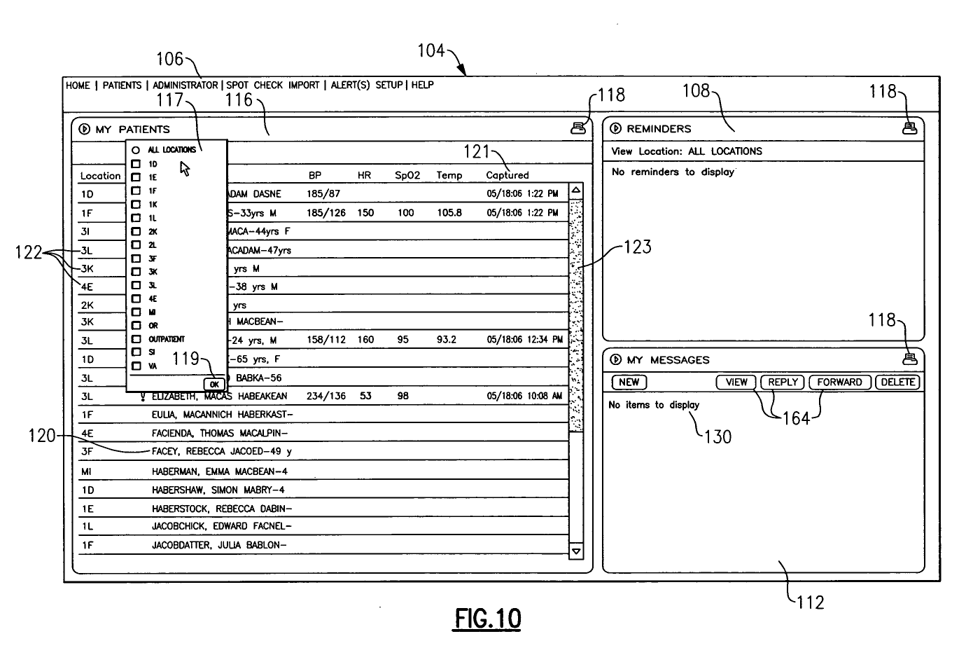

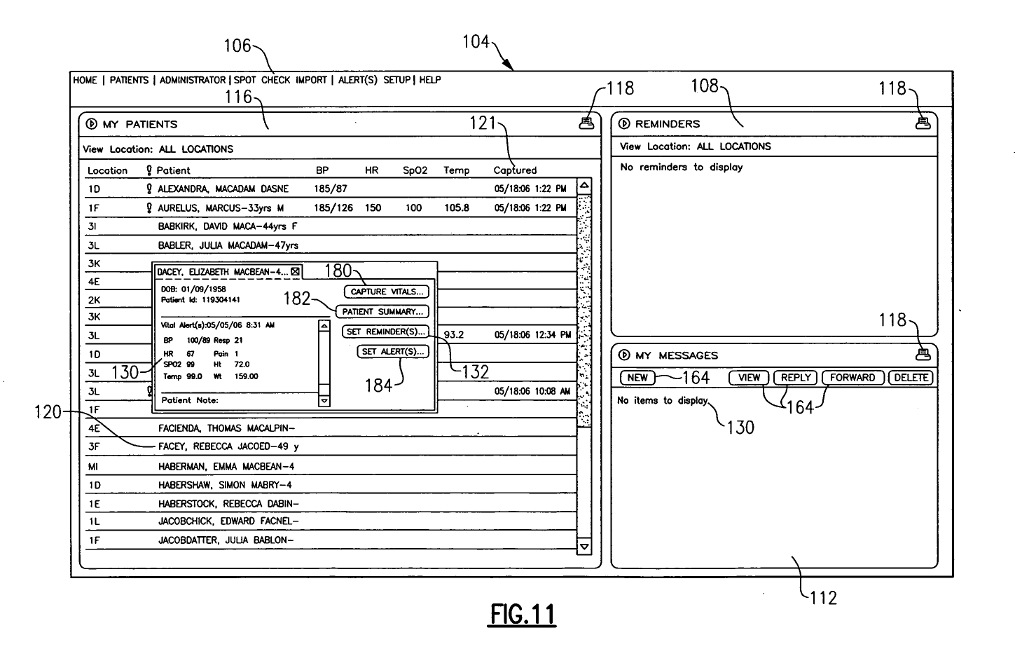

Referring back to FIG. 8, and according to this version, the vitals data that is listed on the list patients window 116 is a summary of the most recent or current vitals taken of the identified patient, including blood pressure (systolic/diastolic), heart rate, SPO₂, and body temperature readings, as well as a date/time stamp 121 indicating when the listed vitals readings were captured. It should be noted in this particular example that ECG data is not utilized, although an ECG assembly is contemplated, such as that shown in FIGS. 1-5. Additionally, the List Patients window 116 lists the unit location 122 ofthe patient in the hospital (e.g., 1D, 2K etc.), as well as data relating to each listed patient, including but not limited to information such as age, sex of the patient and other pertinent demographic data. The selection of patients by location can be changed by clicking onto the location header 125, FIG. 8, of the List Patients window 116 at the top of the patient list 120 and the choice of location can be changed based on clicking on a series of available locations that are provided in a pop-up location selection window 117, FIG. 10, and clicking on an “OK” button 119 located at the bottom of the selection window. The contents of the List Patients window 116 can selectively be printed by selecting the print icon 118 located on the List Patients window 116. Similar print icons 118 are provided on each of the reminders and messages windows 108, 112, as shown in FIG. 10.

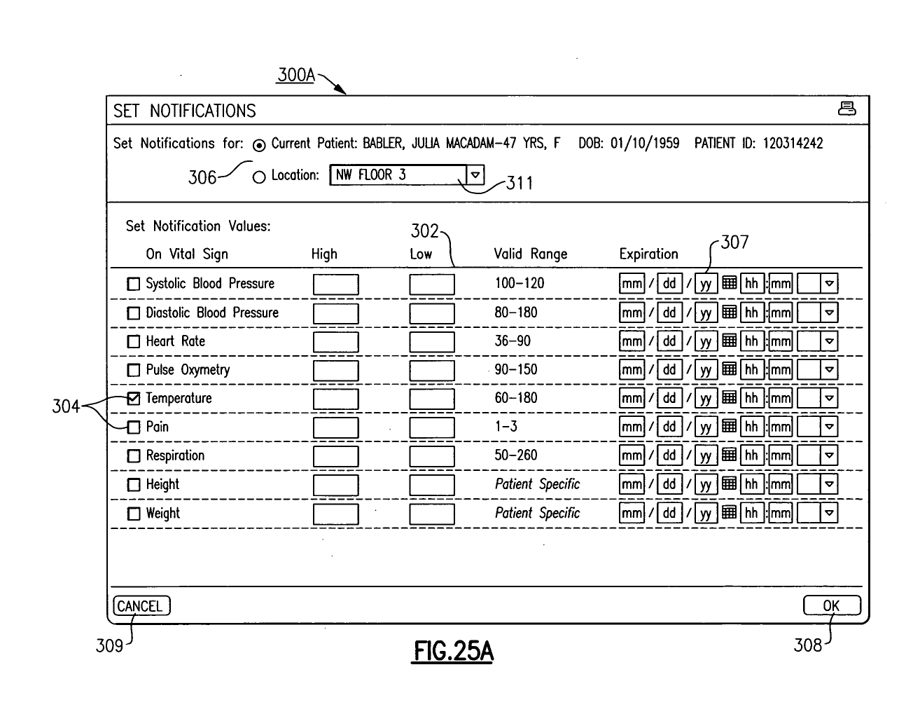

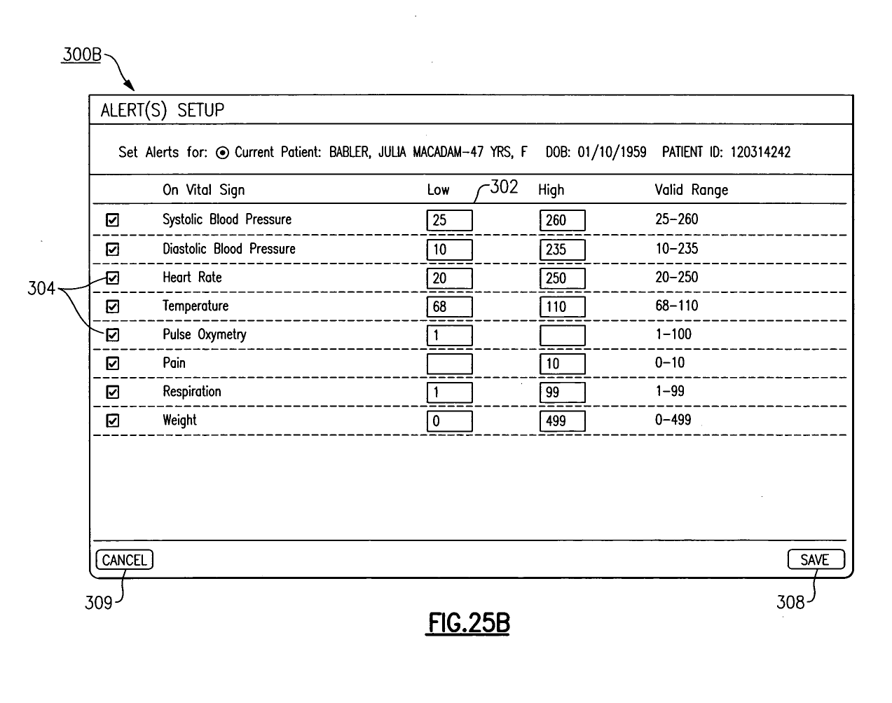

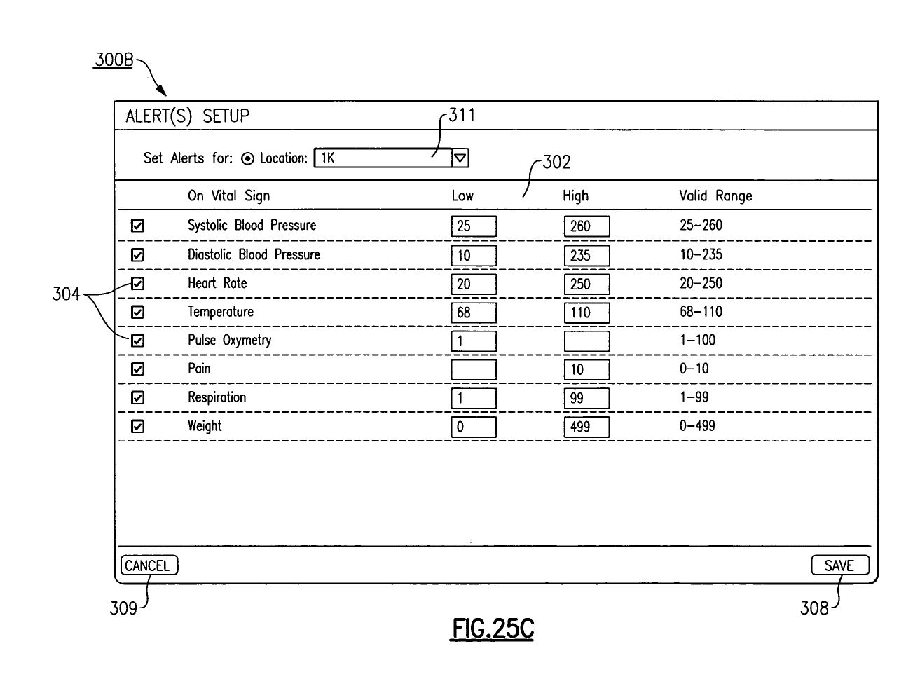

Alert data (also referred to throughout as notification data) can be provided in the tabular list of patients 120 displayed in the List Patients window 116 based on predetermined thresholds in order to assist the user/caregiver. For example, vital signs data that exceeds predetermined thresholds, as set and stored by the memory of the computing device 18 or the vital signs collecting device 26 of the medical diagnostic workstation 10, are shown with a ! symbol next to the listed patient in the list 120. The setup of notifications pertaining to a patient(s) can be preprogrammed into the workstation 10 according to factory settings, but can also be programmed by the user or physician, such as shown in FIG. 25, and described in a subsequent section. According to this example, the names from the tabular list of patients 120 can be presented in bold text to better represent those patients that the caregiver/user has not yet reviewed during the course of rounds. Alternatively, bold and/or colored (e.g., red) indications with regard to any of the listed readings can be used in order to indicate an alert for that patient with regard to the listed parameter. In addition, the ! symbol can be further used to indicate that a patient alert has occurred within a predetermined period of time, even if the most current listed reading is within an acceptable or normal range.

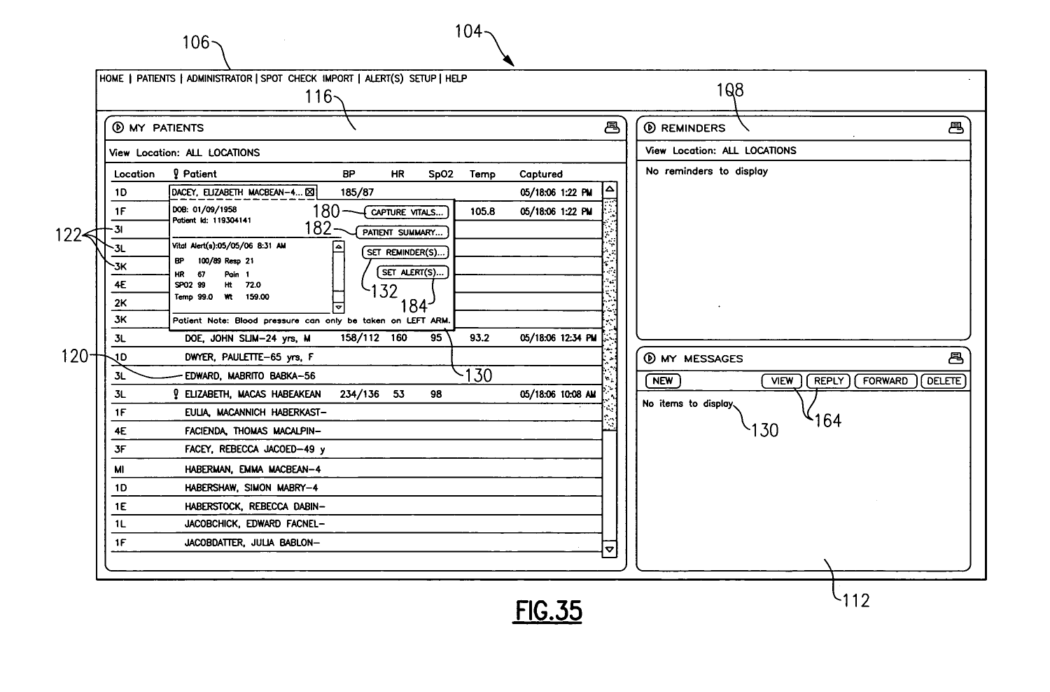

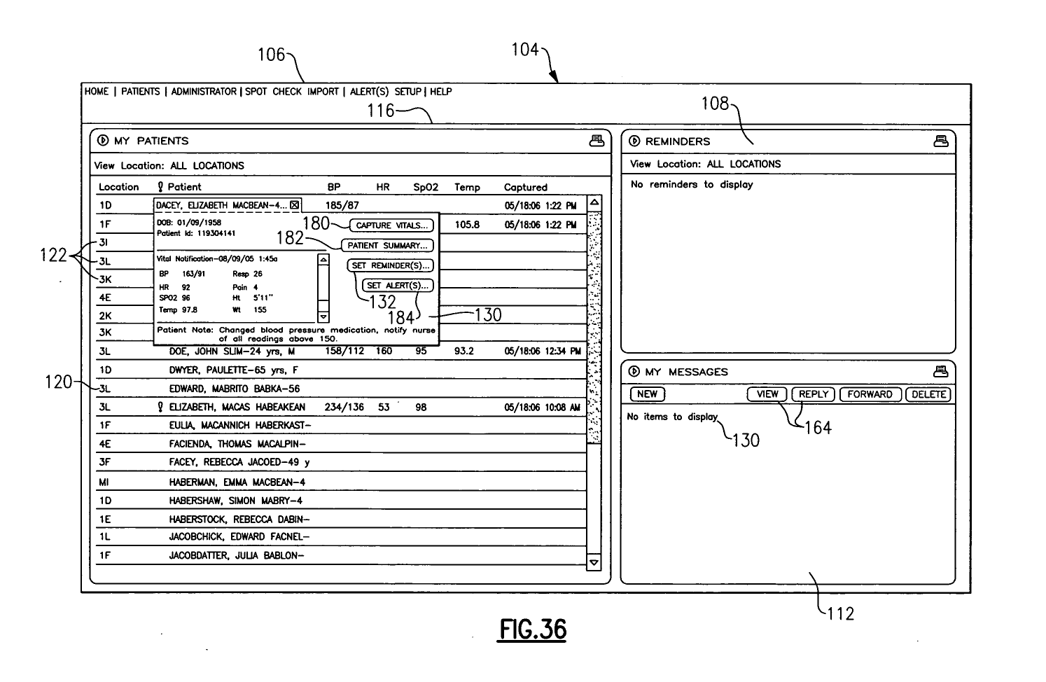

Still referring to the user's display screen 104 of FIG. 8, the Reminders window 108 provides a means for providing information between caregivers of the diagnostic workstation 10 or between caregivers over the computer (e.g. hospital) network with regard to certain patients. In this particularly depicted example, no reminders are listed. Creating a reminder by the user can be performed by at least two techniques. According to a first technique, shown in FIGS. 11-13, clicking directly onto a patient name in the tabular list 120 displayed in the List Patient window 116 causes the display of a “pop-up” patient reveal window 130. The patient reveal window 130 for an identified patient preferably provides more detailed information than that provided in the tabular listing 120 and includes patient demographics, such as sex, date of birth and patient identification number. This reveal window 130 further provides a more complete vitals record than the vitals data listing in the tabular list 120 by additionally listing parameters such as, for example, the most recent respiration, height, weight and pain index measurements, in addition to the already listed vital readings. Additional examples of patient reveal windows 130 are provided in FIGS. 35 and 36. In addition to the above, a listing of patient notes that were taken during vitals capture is also provided at the bottom of the patient reveal window 130, as shown in the examples of FIGS. 35 and 36. It should be noted with regard to vitals capture, that this part of the workflow is described in greater detail in a succeeding portion. Finally, the patient reveal window 130 also includes a series of action or navigation buttons on the right hand side of the window. Included among these navigation buttons is a reminders button 132 as well as a capture vitals button 180, a patient summary button 182 and a set alert(s) button 184.

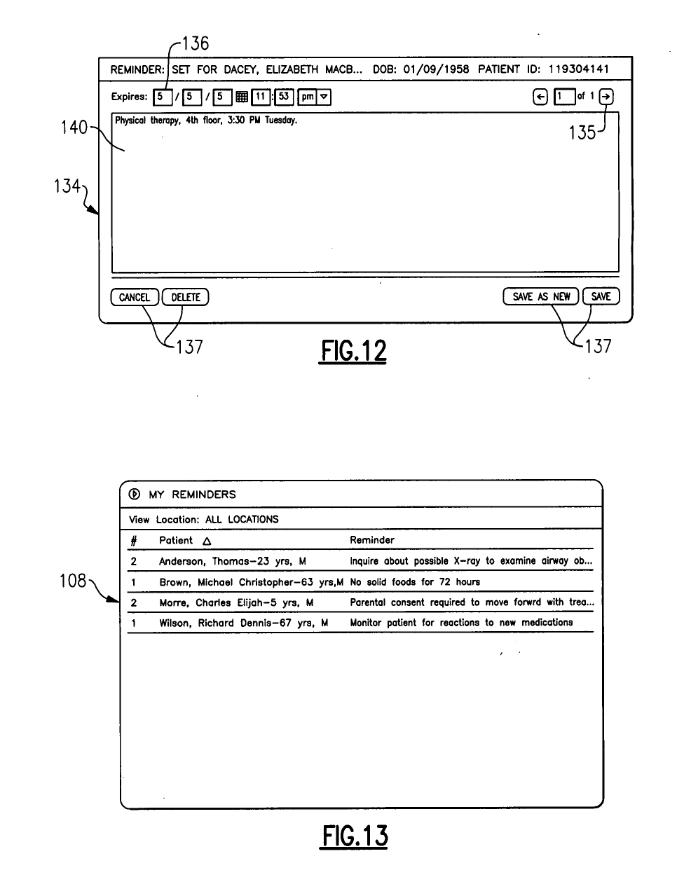

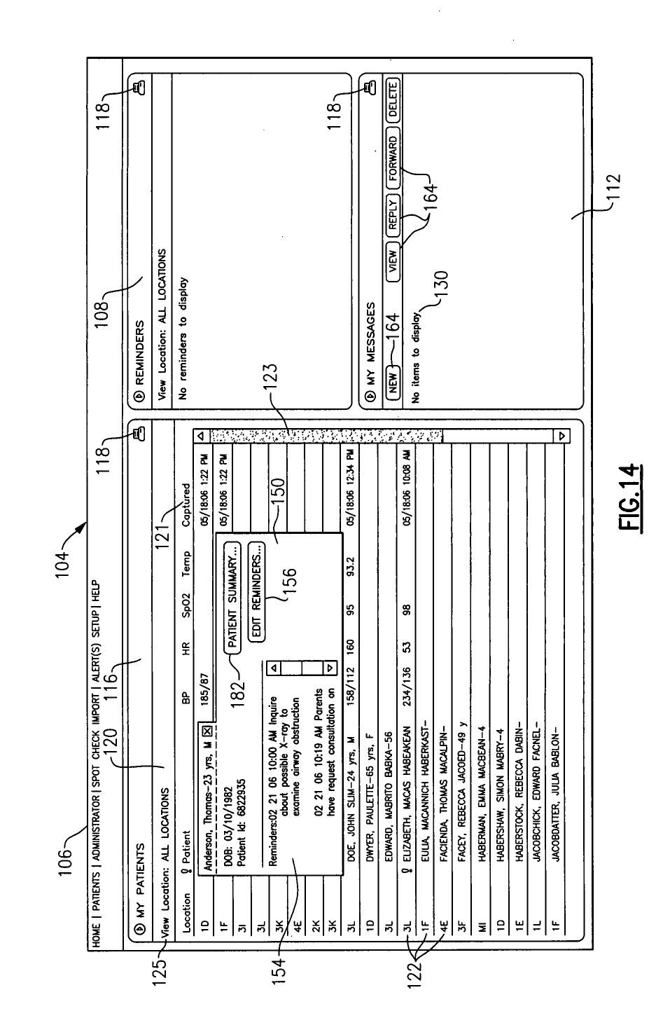

According to this embodiment, clicking onto the set reminders button 132 pulls up a set reminders display window 134 for the identified patient, an example of which is depicted in FIG. 12. The format of the set reminder window 134 includes patient information, including the patient's identification number at the top of the window, as well as a separate field 136 that includes an expiration date that can be set for the reminder if no action is taken using a pick list. The workstation 10 according to this specific embodiment is ordinarily programmed to include a default expiration time of 12 hours, as measured from the time the reminder is set. A text field 140 includes an existing message or allows a new or edited message to be created therein. Reminders for the identified patient can be sequentially scrolled using arrows 135 provided in the upper part of the window 134. The workstation 10 is configured to erase or deactivate the reminder following the expiration date. A series of action buttons 137 are also provided in the set reminder window 134, including a cancel button, a delete button, a save button and a save as new button, respectively. The save as new button, when selected, creates a new reminder while the save button overwrites the current reminder, this reminder being added to the overall reminders list, a further example of the latter being shown in FIG. 13. The cancel and delete buttons are used for selectively canceling or deleting the reminder in advance of the expiration date. Clicking on the set reminder button 132 of the patient reveal window 130, FIG. 11, when no reminders are present for the patient will also open a set reminders window 134, allowing reminders to be created and listed in the Reminders window 108, as shown for example in FIG. 13.

Reminders can also be accessed for a particular patient directly through the Reminders window 108 ofthe user's display screen 104 by clicking on a patient listing in the window using the mouse 38, FIG. 3, or other input device prompting a reminders reveal window 150, an example of which is shown in FIG. 14. The reminder reveal window 150 according to this embodiment includes specific patient demographic information, as well as a complete list of reminders pertaining to the identified patient. An edit reminders button 156 provided in this reveal window 150 that, when selected, toggles each of the sequentially scrolled set reminder windows 134 of FIG. 12 pertaining to the listed reminders or permitting modification and/or editing or creating of new reminders.

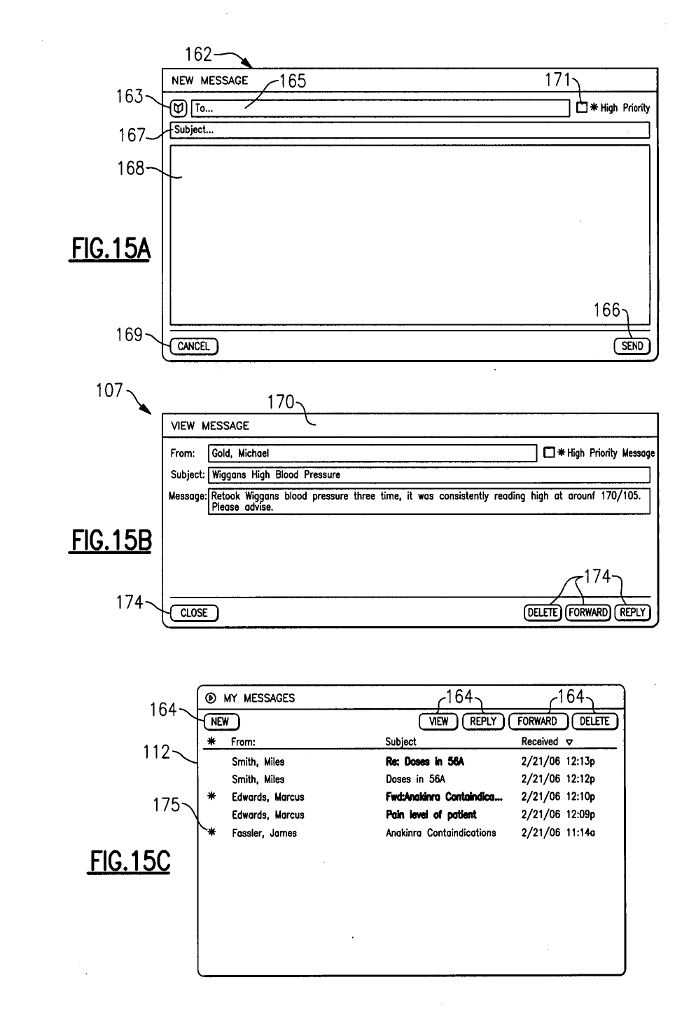

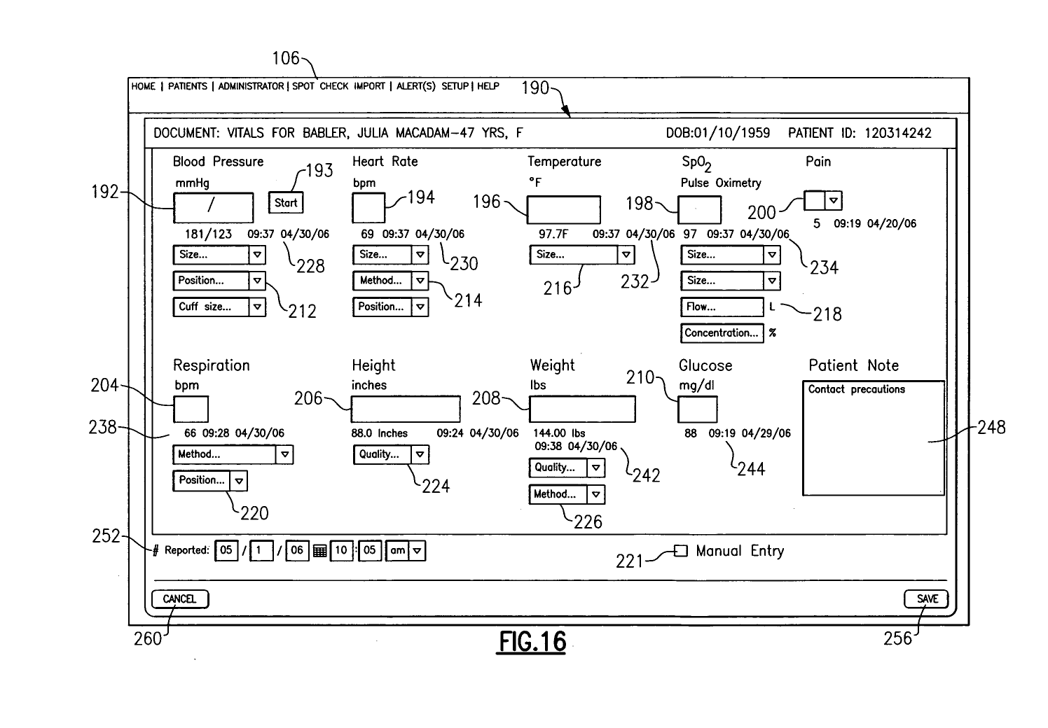

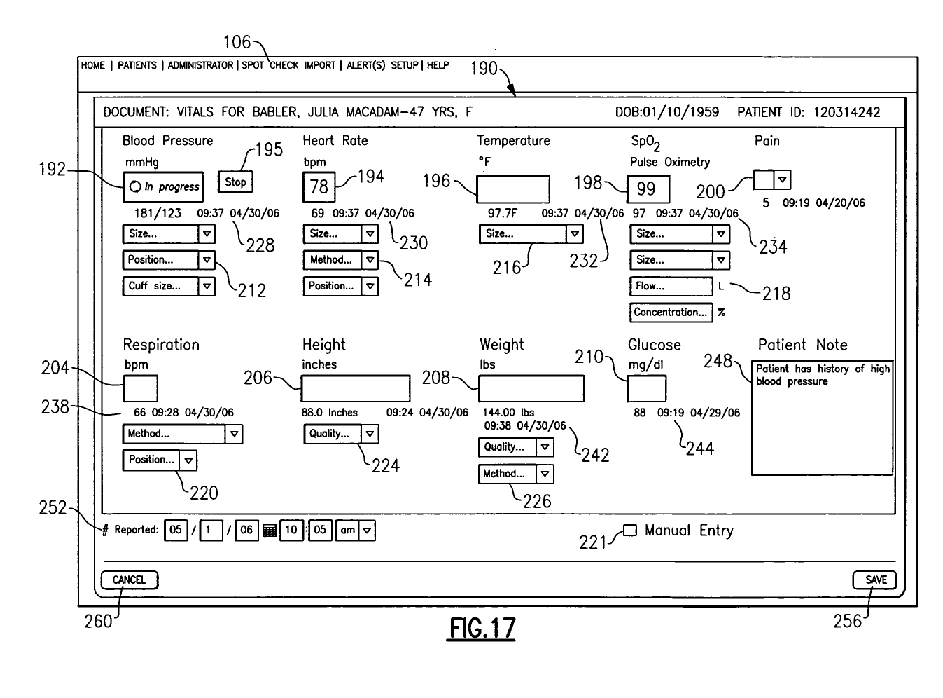

Referring back to the user's display screen of FIG. 8, the Messages or inbox window 112 includes a listing of messages 160 that have been received by the diagnostic workstation 10 between users. In this embodiment example, no messages are displayed. These messages can be accessed for reading by way of specific action buttons 164 that are arranged above a list of messages 160 in the Messages window 112. The action buttons 164 are accessed by first highlighting a message of interest, wherein the message can then be replied to, forwarded, or deleted. Among the action buttons 164 is a New button that, when actuated, displays a new messages window 162, an example of which is shown in FIG. 15(a). The new messages window 162 includes an address book icon 163 at the upper left hand portion of the window. Clicking on the icon 163 displays an address book (not shown) to permit a note recipient to be searched. Alternatively, the recipient's name(s) can be entered manually into a To data entry field 165. This window 162 further includes a subject line field 167 and a message text field 168. A cancel button 169 enables a user to selectively cancel the new message and navigate back to the user's display screen 104, FIG. 8. A send button 166 permits the message to be sent to the intended recipient(s). The new messages window 162 further includes a high priority selection field 171, that when checked by the user marks the message as a high priority message.

A sample message is shown in FIG. 15B, as displayed in a view message window 170, which pops up in the user's display screen 104 when a message is selected. The view message window 170 also includes a series of buttons 174 enabling further action (e.g., close message, deletion, forwarding, etc) relating to the message therein. Each message listed in the messages window 112 is listed, according to this embodiment and shown by way of example in FIG. 15(c), by way of the sender, message subject, and time of the message. As previously noted, urgent messages (shown in this figure with asterisks 175) can also be given a priority status and can be highlighted as shown. According to one version, the workstation 10 will not power down until the user has acknowledged the priority messages. The messages can be received by the diagnostic workstation 10, for example, through a standard web-based Internet or other network connection, such as from another diagnostic workstation 10, a central monitoring station (not shown), or other remote site using a web browser or alternatively via a VOIP (Voice over Internet Protocol), a pager, from devices in which readings could trigger a message, cellular phone text messages, and the like. The remaining action buttons 164 permit the user to view (see FIG. 15(c) and FIG. 8), reply, forward or delete any selected messages.

Referring briefly to FIG. 11, and with regard to a specifically identified patient, it was previously noted that highlighting a listing in the patients list 120 in the List Patients window 116 of the users display screen 104 causes a patient reveal window 130 to be pulled up for the selected patient. Among the options that are available on the patient reveal window 130 is a capture vitals button 180.

Referring to FIGS. 16-20, a capture vitals display screen 190 is accessed and displayed on the display 30, FIG. 1, of the diagnostic workstation 10, FIG. 1, upon clicking upon the capture vitals button 180 on the patients reveal window 130, FIG. 11. The workflow of vitals capture using the dedicated workstation 10 and other devices/apparatus will now be described. An exemplary capture vitals display screen 190, as shown in FIGS. 16-20, includes the global header 106 at the top of the display screen, as well as a series of data entry windows 192, 194, 196, 198, respectively, for data entry of specific physiologic parameter readings (in this instance, blood pressure, heart rate, temperature and SpO₂), as measured by the physiologic sensors that are associated with the vital signs collecting device 26 that is attached to the medical diagnostic workstation 10 and connected to the computing device 18. The computing device 18 is configured to collect and properly insert the appropriate physiologic readings in the corresponding data entry windows when the sensors of the collecting device 26 are attached to the patient and the resident vital signs measuring device 26 is activated, such as through the user interface thereof. Alternatively, the diagnostic workstation 10 also permits manual entry of physiologic parameters for purposes of vital data relative to the capture vitals display screen 190. The latter is done in which users click upon a “manual” button 221 and then text can be typed into each field. Users can also change the time of the reading—for those readings taken previously by means of a time entry field 252, located at the bottom of the capture vitals display screen 190.

Turning more specifically to FIG. 16, the capture vitals display screen 190 according to this embodiment further includes a plurality of corresponding data entry windows for other physiologic parameters such as pain 200, rated as an index from a scale of 1-10, respiration rate 204, height 206, weight 208 and blood glucose 210, as well as corresponding windows for the selection of qualifier data that pertains to each of the above parameters. The vitals capture screen 190 further includes a Notes entry window 248, enabling manual entry of text notes, as needed, by the user, such as by using the keyboard 34, FIG. 1.

As noted according to this particular example, vitals data entered in each of the data entry windows 194, 196, 198 can be obtained directly from the vital signs collecting device 26, to permit timely entry of heart rate, body temperature, and pulse oximetry upon attachment of the sensors of each parameter module to a patient and activation of the vital signs collecting device 26. The measurement of blood pressure is selectively initiated by depression of a start button 193 located on the vitals capture screen 190 adjacent the data entry window 192. Clicking on the start button 192 using the mouse 38, FIG. 1, the keyboard, FIG. 1, or other input device initiates a signal to activate the pump (not shown) of the vital signs measuring device 26 and inflate the cuff (not shown), in that blood pressure is not a continuously measured from the moment the sensors are attached to a patient. That is to say, blood pressure is a selectively measured parameter. Alternatively, the user interface of the vital signs collecting device 26 can be used to initiate a blood pressure measurement cycle. In addition and referring to FIG. 17, a message appears in the data entry window 192 that a blood pressure measurement is in progress. A stop button 195 also appears during the blood pressure measurement cycle that can be selectively clicked upon to terminate the measurement. The remaining data, such as that relating to respiration rate, weight, pain, and other parameters, can be captured by separate apparatus that is not directly connected to the workstation 10 or is obtained through other means (asking the patient, for example, with regard to pain index), wherein this resulting data is entered in each window directly using an input device (e.g., the keyboard 34) of the diagnostic workstation 10. As previously noted, data can also be imported to the workstation 10 from a vital signs collecting device 26, such as Spot Vital Signs Lxi device 43, FIG. 6, via a wireless connection with the workstation, the latter being equipped with an antenna to receive data. The importation of data from this device is described in greater detail below.

As to the qualifier data and in terms of providing a better definition therefore, this latter form of data, as discussed herein and according to this exemplary embodiment, includes blood pressure qualifiers 212, including the limb to which the cuff is attached (left arm, right arm, left leg, right leg), the position of the patient (standing, lying or sitting), and the cuff size (whether a neonate cuff (1-5), a newborn cuff, an infant cuff, a small child cuff, a small adult cuff, an adult cuff, a large adult cuff, or a large long adult cuff). Heart rate qualifiers 214 include the site (whether left or right side of the patient), the method used to take the heart rate (ausculate, Doppler or palpated), and the position of the patient (whether lying, sitting or standing). Temperature qualifiers 216 include the location or site of the temperature measurement (whether axillary, rectal, oral or tympanic). Pulse oximetry qualifiers 218 include the method of measurement (aerosol/humidified mask, face tent, mask, nasal cannula, non rebreather partial rebreather, T-piece, tracheostomy collar, ventilator, venturi mask, room air, or oxymizer), location of the oximetry measurement (finger, ear), the flow rate and concentration. Typically, the oxygen content range is in the range of about 1 to 20 liters and the concentration is about 21 percent to 100 percent. In each of the latter instances, the user can also manually type in the specified value in the provided window 218. Respiration qualifiers 220 include the method used (assisted ventilator, controlled ventilator or spontaneous) and position of the patient (lying, sitting or standing). Height qualifiers 224 include the quality of measurement (whether actual or estimated). Weight qualifiers 226 include the quality of measurement (whether actual, dry or estimated) and the method of measurement (bed, chair or standing). Finally and though not shown, pain qualifiers can also be provided. For example and according to one aspect, pain qualifiers can be provided to indicate whether any action or treatment has been taken as a result of a specific pain index (repositioning of patient, medication delivered to patient, no action taken, etc) has been effective in treatment.

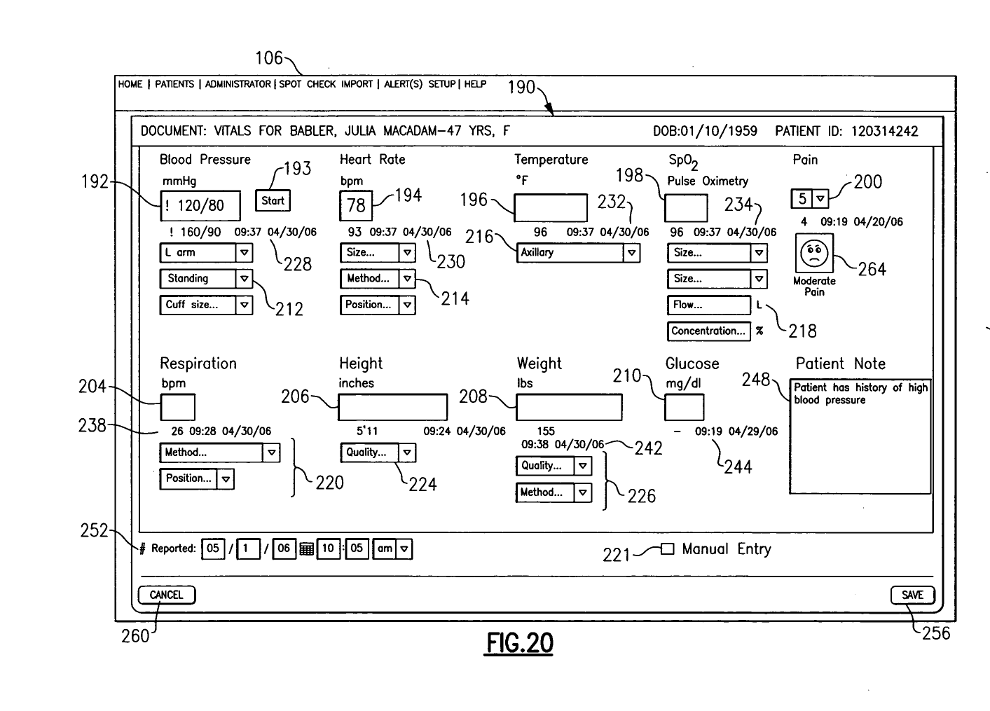

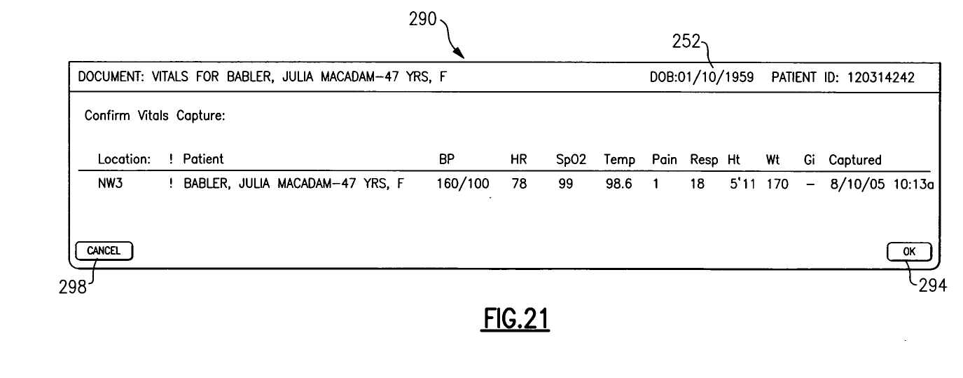

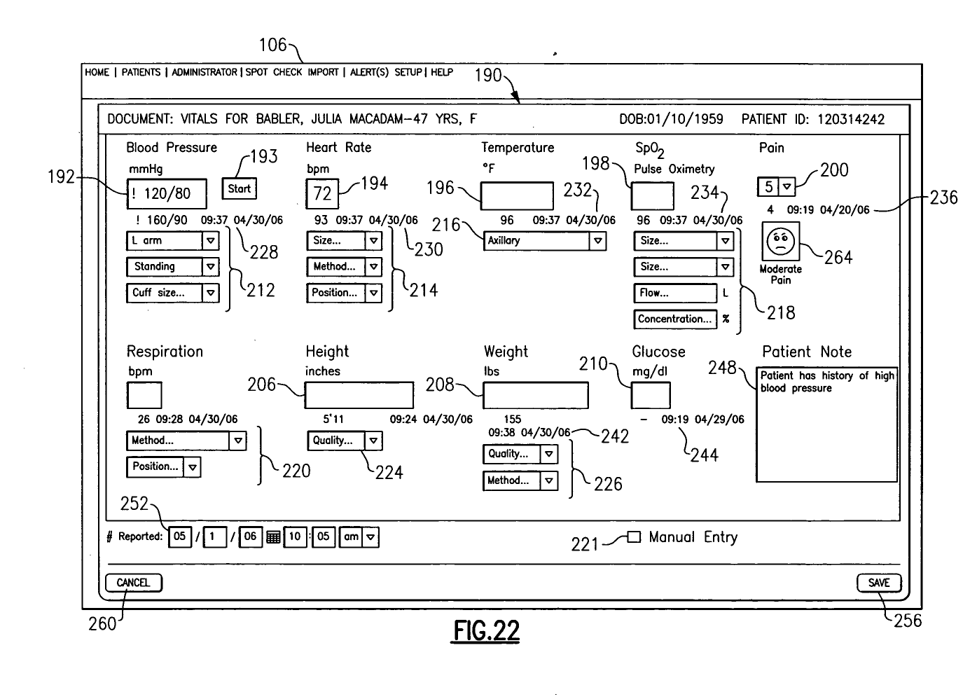

Referring to FIG. 20, an alternative graphical icon 264 representative of the pain index (low, moderate or high) is shown. Qualifier data is believed to be an important part of the patient summary record and adds comprehension in that the qualifier data is representative of information related to a medical device, such as blood pressure, but is not collected directly by the device. Still, this data is important in understanding the overall status of the patient. In addition to the above, the capture vitals display screen 190 also lists, beneath each data entry window, the most recent reading for each parameter listed herein as 228, 230, 232, 234, 236, 238, 240, 242, 244, in relation to each corresponding data entry window. Similarly to the denotations in the patients window 116, bold face and colors are used herein to indicate readings that exceed a predetermined threshold or acceptable range of values while a ! symbol indicates that a reading has been taken within a predetermined amount of time that has exceeded a predetermined threshold. The date/time of measurement can be selected via the time entry field 252 that is provided in a lower portion of the screen 190, the field including a pick list to set both date and time of the measurements.

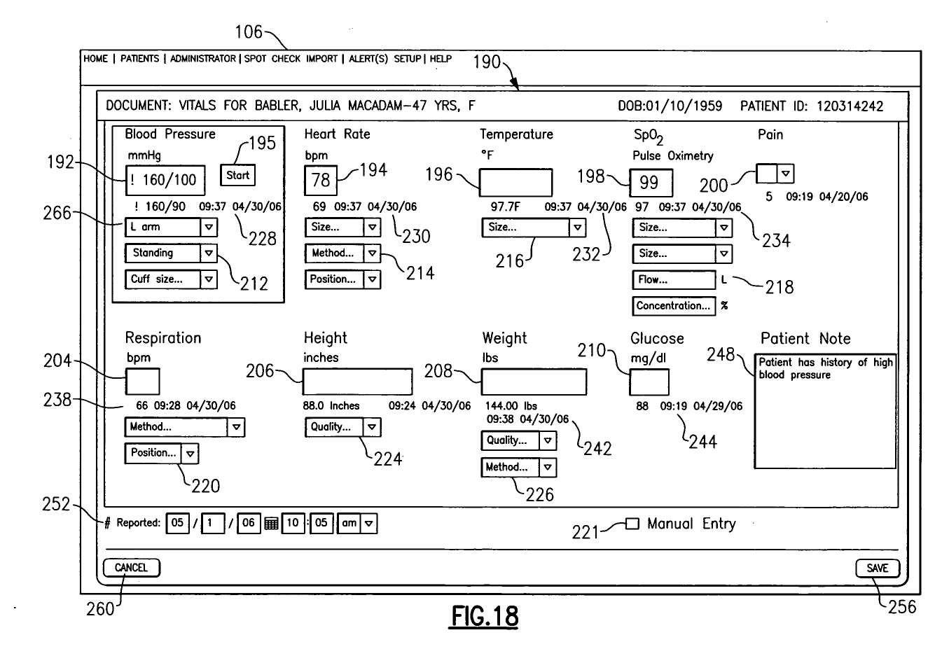

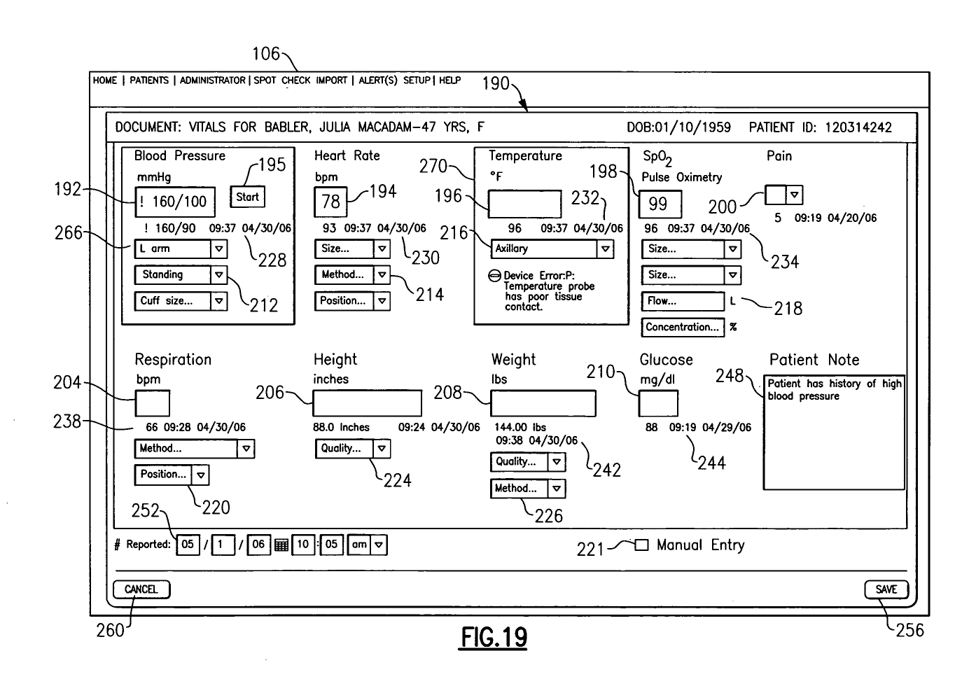

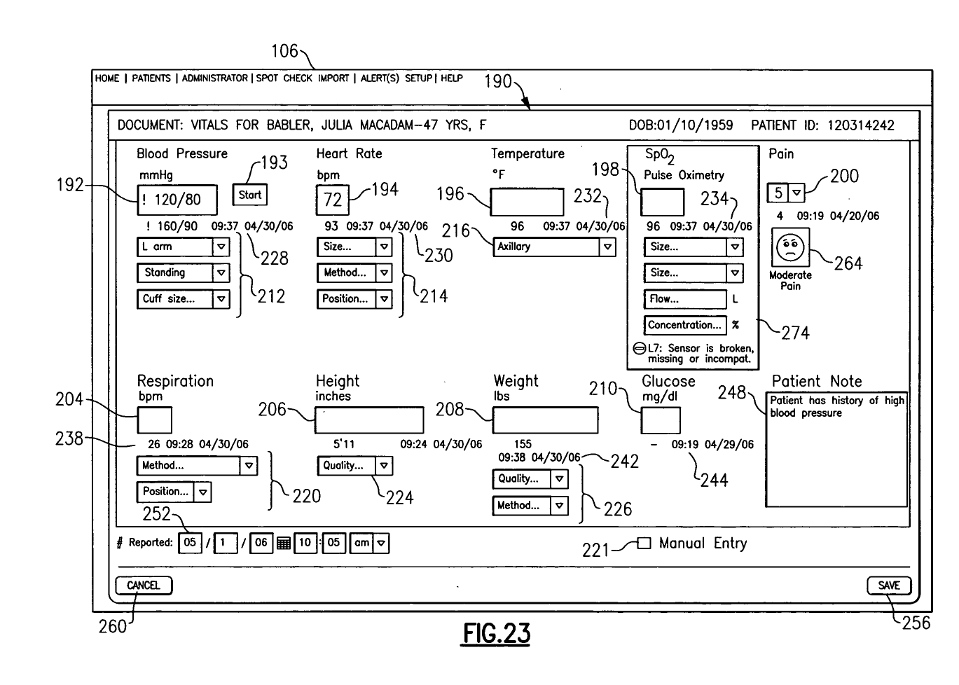

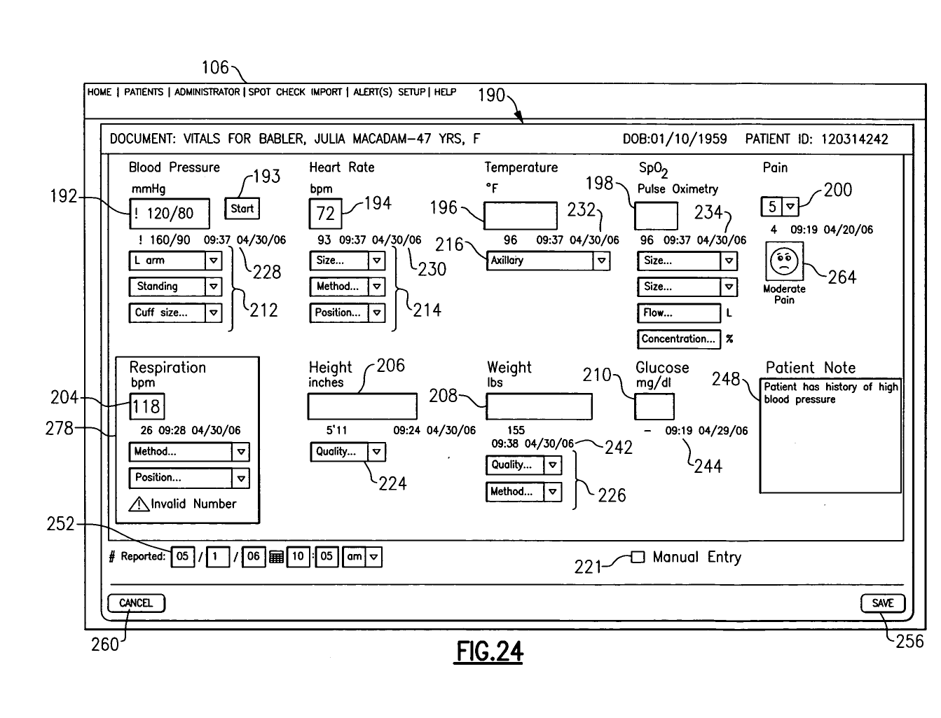

According to this embodiment and if a reading(s) exceeds a predetermined threshold or there is an equipment problem or failure, the corresponding data entry window and qualifier data portions of the capture vitals display screen 190 are highlighted. For example, and as shown in FIG. 18, a blood pressure reading for the identified patient has exceeded a predetermined threshold, as indicated by the highlighted box 266 and further indicated by a reading in bold face in the data entry window 192. A further typical example is provided in FIG. 19 in which a device error is noted in temperature measurement. In this example, a highlighted box 270 indicates an anomaly relating to specific equipment error. In terms of equipment errors, the computing device 18 can be configured to display the error messages of the measuring device 26 or other faults, such as broken leads, loss of sensor connections, low power, or other conditions. Alternative examples are shown in FIGS. 22-24 for another vitals capture display screen 190. In this example, vital data is entered for blood pressure, heart rate and pain. Referring specifically to FIG. 23, an equipment error with regard to the pulse oximetry module of the vital signs measuring device 26 is noted by means of a highlighted box 278 indicating that the oximeter sensor is broken. The workstation 10 is programmed using this and other windows/boxes to alert the user to a number of equipment/measurement errors. FIG. 24 illustrates another example of a user alert that is caused by a possible transcription error in posting a respiration rate wherein a reading of 17 has apparently been incorrectly entered as 117. This and other similar types of error are noted to the user in a highlighted box 278, thereby permitting modification prior to final entry and confirmation as will now be described.

As shown in each of FIGS. 16-20 and 22-24, the capture vitals display screen 190 further includes a save button 256 and a cancel button 260, each located at the bottom of the screen, according to this specific embodiment. Depression of the cancel button 260 using an input device cancels the entire set of readings. Individual vital signs readings for the patient can be repeated or overwritten until users obtain a desired results. Cancellation of individual readings can also be done, as follows: For example, blood pressure readings can be cancelled in the middle of a measurement cycle by actuation of the stop button 195, FIG. 17. Temperature readings can be cancelled by removing the temperature probe from the target site (e.g., the mouth) and an SpO₂ reading can be cancelled by removing the probe 84, FIG. 1 from the finger or other peripheral patient site. Actuation of the save button 256 causes a confirmation pane or vitals capture window 290 to simultaneously display in overlaying fashion or “pop-up” onto the capture vitals display screen 190, as shown in FIG. 21.

The confirmation of the data presented by the confirmation of vitals capture display window 290 includes specifics about the patient, including date of birth and patient identification number, as well as the captured data without the qualifiers. A date stamp and patient location are also provided according to this example window. Bolded data readings include those readings that have exceeded a predetermined threshold or a range of acceptable readings. Pressing an “OK” button 294 located at the bottom of the confirmation window 290 permits all of the captured readings to be saved into the memory of the diagnostic workstation 10 as part of the permanent record, while a cancel button 298 also located on pane 290 permits cancellation of the data. Though not shown, this window can further include a modify button wherein part of the vital signs data can be modified and then retaken, as needed. Confirmation is made by selecting the ok button wherein this confirmation creates a “sign-off” of vitals that are captured using the diagnostic workstation 10, further enabling the workstation to track the user(s) who actually took the readings. In addition, the requirement for confirmation provides safeguards in tracking clinical review of data by a nurse or physician.

As previously noted herein, the user of the herein described diagnostic workstation 10 can be alerted or notified when patient readings exceed a predetermined threshold or acceptable range of values. Notifications or alerts can be set universally for all patients or can set either on a group (floor, ward, etc.) or an individual patient to patient basis according to another aspect of the present invention. By clicking on the ALERT SETUP option in the global header 106, FIG. 8, at the top of the user's display screen 104, FIG. 8 (or other selected display screen, see for example, the display screen 310 of FIG. 26), a set notifications display screen 300 can be accessed. Exemplary set notifications display screens 300A, 300B are shown in FIGS. 25(a)-25(c). Each set notifications display screen 300A, 300B includes a set notifications values window 302, listing each of the parameters that are measured by the diagnostic workstation 10. A range (high/low) of acceptable values can be selected by the user for individual parameters using this window 302, as selected through a series of corresponding parameter selection check boxes 304. The listing further provides a typical valid range for each listed parameter, serving as a default if notifications are not otherwise set by the user. Each of the exemplary set notifications display screens 300A, 300B according to this embodiment further permit alerts for either a current patient or for a specific location through appropriate selection windows 306 at the top of the display screen. Each display screen 300A, 300B also includes appropriate buttons 308, 309 enabling the user to either verify or cancel selections, these buttons being located at the bottom of the screen. As noted, two exemplary versions of a set notifications display screen are shown. In the first version 300A, shown in FIG. 25(a), either of the current patient and/or patient location can be selected for the setting of notifications, the latter selection being done through a scrollable drop-down box 311. In this version, the expiration date and time can also be individually selected using a date field 307. In the other exemplary version 300B, FIGS. 25(b) and 25(c), separate display screens 300B are provided to set each of the current patient and location as opposed to the single screen version of FIG. 25(a).

According to another aspect, each of the patient reveal windows 130, FIG. 11, and the reminders reveal windows 150, FIG. 14, include a view patient summary button 182. Clicking on this button 182 on either reveal window 130, 150, causes the presentation of a patient summary display screen 310, an example of which is shown in FIG. 26, for an identified patient. The patient summary display screen 310, according to this particular embodiment, includes a series of windows or panes disposed thereupon that comprises a clinical summary window 320, a problems list window 330, an allergies and sensitivities window 340, and a patient notes window 350, respectively. A global header 106 is also provided to permit navigation to and away from this display screen. This global header, as shown in FIGS. 26-28(a) is different than that of FIG. 31 (and also FIG. 8). The functions of the listed options are roughly equivalent and the differences are shown for purposes of example.

In addition, the patient summary display screen 310 further optionally includes an image 312 of the patient disposed at the top of the screen, according to this particular embodiment, as well as specific patient-related data (i.e., date of birth (DOB), patient identification number and primary care physician (PCP)) also provided at the top of the patient summary display screen 310 in the vicinity of the patient image. The image 312 of the identified patient can be obtained, for example, from the scanner 50, FIG. 1, when a patient record is initially created for the workstation 10. New details concerning the patient typically come from the ADT messages that show a patient that has been admitted. Each of the data windows of this exemplary patient summary display screen 310 will now be described in greater detail, referring herein to FIGS. 26-31.

In brief, the clinical summary window 320 provides selectable listings of capture vital data that has been taken for the identified patient. FIG. 26 provides a default listing of patient data as set by the diagnostic workstation 10. In this format, current visit data is posted for the identified patient in a tabular flowsheet format 323. As in the preceding, readings with bold face are shown to alert the user to readings that have exceeded a predetermined threshold or acceptable range of values. The current view can be printed by a user by selecting a print icon 328.

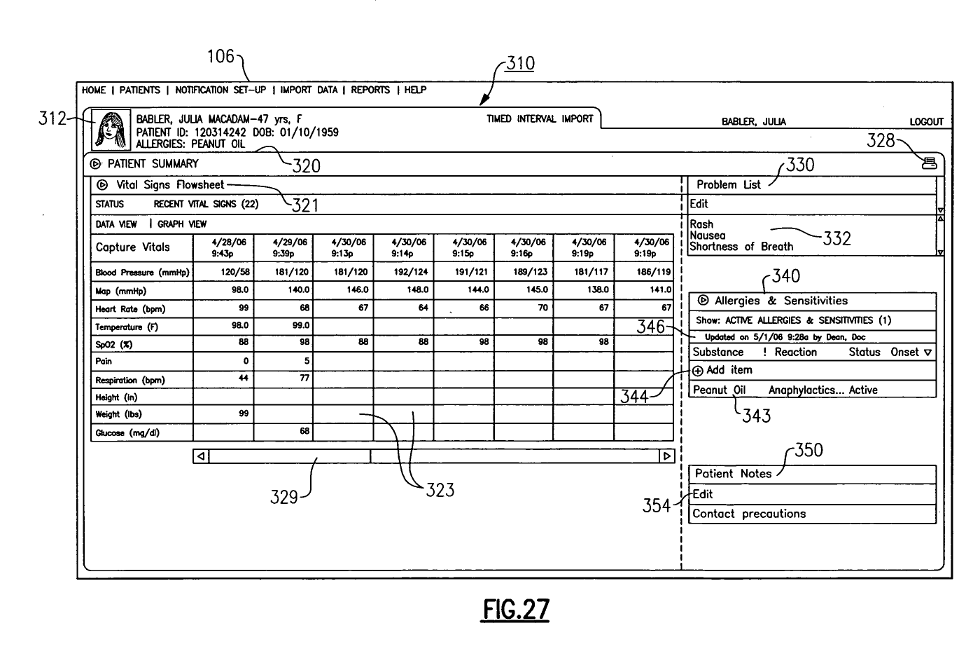

According to FIGS. 26 and 27, the tabular flowsheet format 323 of the vital sign and other readings concerning the patient includes a listing of the capture vitals (i.e., blood pressure (systolic/diastolic), mean arterial pressure (MAP), heart rate, SpO₂, body temperature, respiration, pain, weight, height, Body Mass Index (BMI) and blood glucose) for a specified number of readings are depicted in tabular form. In this example, the eight (8) most recent readings taken over the course of 48 hours are actually displayed, though the entire tabular summary of readings relating to the identified patient can be accessed, through use of a horizontal scrolling bar 329. In this particular example, 568 separate records/sets of readings relating to the identified patient can be accessed, though this latter parameter can be easily varied. The amount of data that can be selected for display can be further augmented by a pop-up data display window 326, FIG. 31, which is selected at the top of the header relating to the amount of data that is being displayed. In the present example, only data relating to the current patient visit is displayed for scrolling. Additional choices are selectively available to broaden or narrow the amount of patient data presented using the window 326.

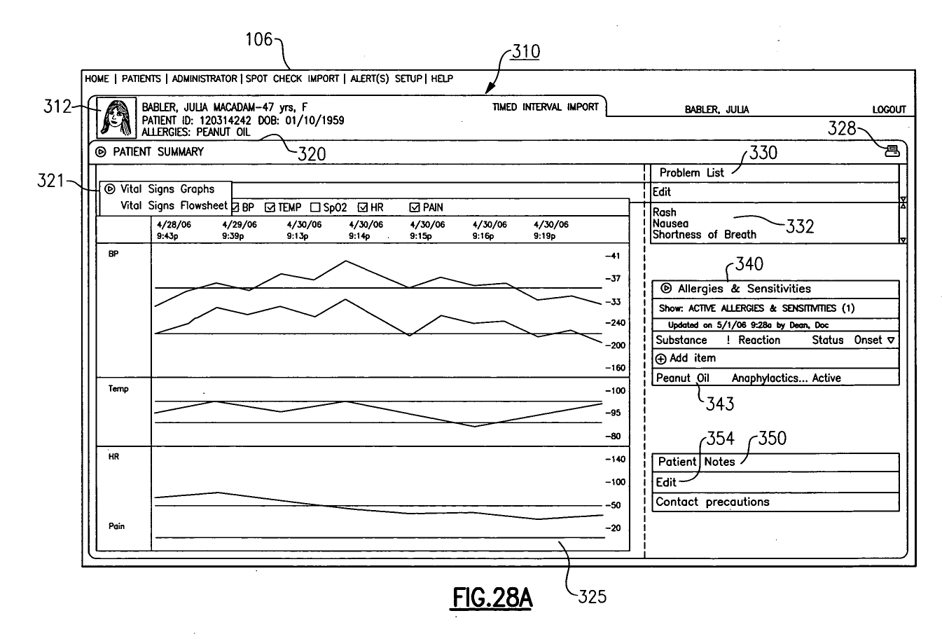

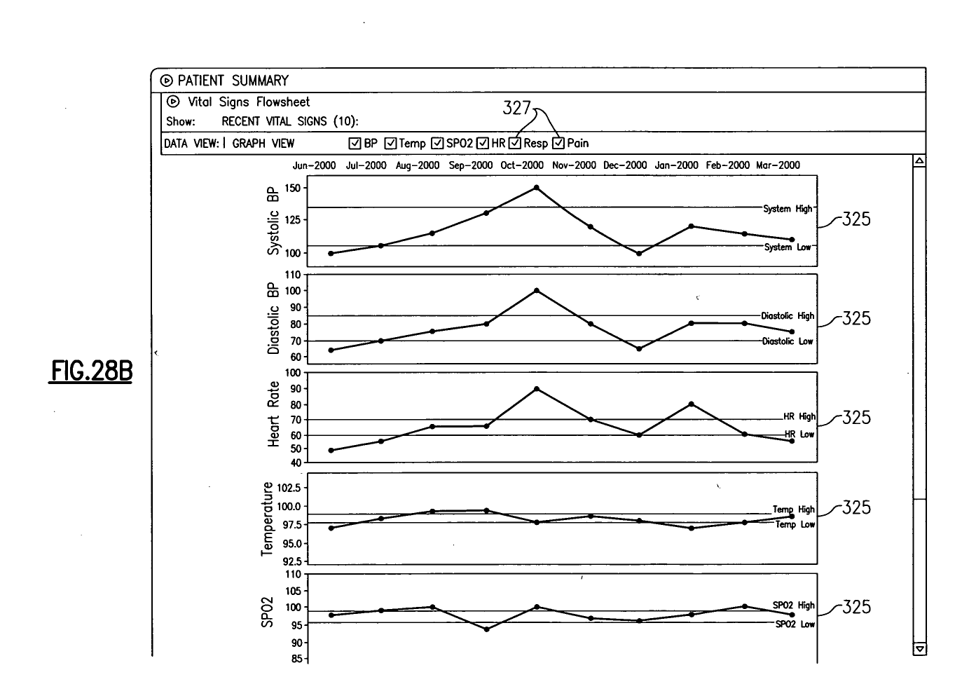

The format of data that is presented in the clinical summary window 320 can also be selectively changed by the user. Referring to FIGS. 28(a) and 28(b), graphical as opposed to tabular displays of the identified patient's data can be selectively displayed. According to this example, a pop-up data format window 321, FIG. 28(a), is available to change the format from a tabular flowsheet to a graphical flowsheet. Specific parameters can be selected for display by the user using a series of graphical selection boxes 327 located at the top of the window 320. According to this specific example, blood pressure, body temperature, heart rate and pain index are depicted by way of checking appropriate parameter check boxes at the top of the vital signs graphs header. Examples of graphical data flowcharts 325 are illustrated in FIGS. 28(a) and 28(b).

Additional details related to any single set of readings can be obtained by clicking onto the time and date portion listed above the tabular set of readings, thereby extracting a simultaneously displayed or “pop-up” patients summary reveal window 324, an example of which is shown in FIG. 30. The patients summary reveal window 324 includes patient demographics, as well as qualifier data relating to the parameter readings, which are also listed in this window. The readings can be selectively edited or removed from storage by clicking an appropriate remove button 318 located in the patients summary reveal window 324.

As noted above, and in addition to the clinical summary window 320, the patient summary display screen 310 further includes three (3) additional windows or panes that are used for patient related problems, allergies and sensitivities, and patient notes, respectively. As shown in FIGS. 26, 27 and 31, the problems list window 330 located in the upper right hand corner of the patient summary display screen 310, according to this embodiment, includes a text field 332 that can be edited to add information about the patient's complaints and conditions, such as, for example, whether the patient is a diabetic. According to this embodiment, the problems list is a single continuous entry that is viewable by all users of the workstation 10.

Referring to FIGS. 26-31, the allergies and sensitivities window 340 located beneath the problems list window 330 permits display and entry of an identified patient's allergy information, the window listing a tabular summary of allergy related information that includes, according to this embodiment, the allergen or substance, patient reaction, status of the allergy (e.g., active or inactive) and date of onset of the allergy, as listed items that are provided or which can be added to a table field 343. Additional items can be added to the tabular listing or summary by clicking onto an Add item icon 344 and manually entering the information into the table field 343. A review fill-in box 346 permits the user to indicate that patient allergy information has been reviewed by checking an appropriate box provided in the window 340, using the mouse 38 or other input device of the diagnostic workstation 10.

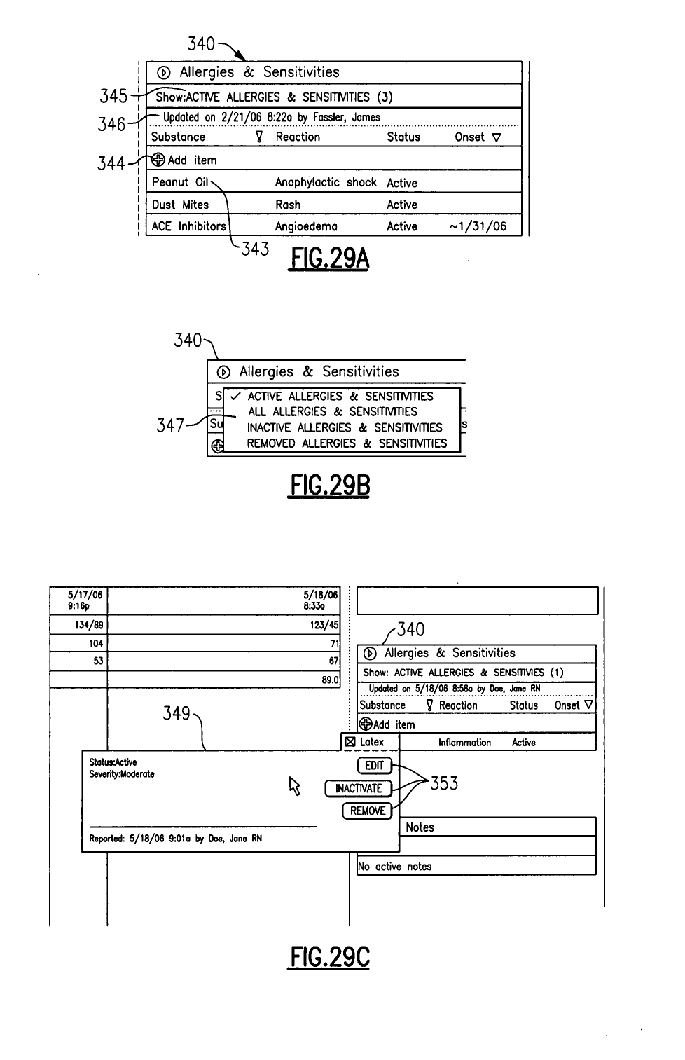

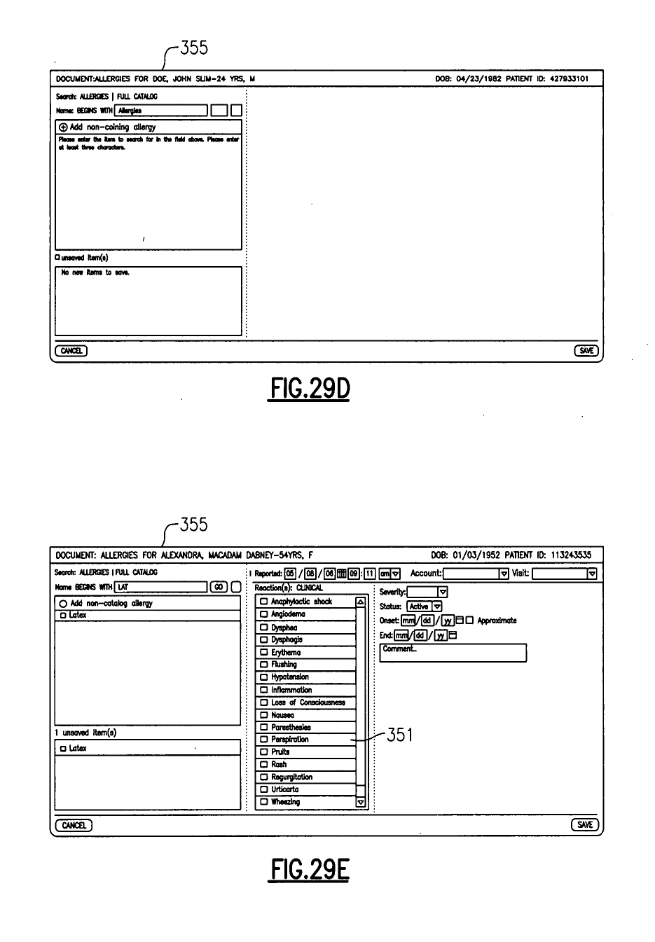

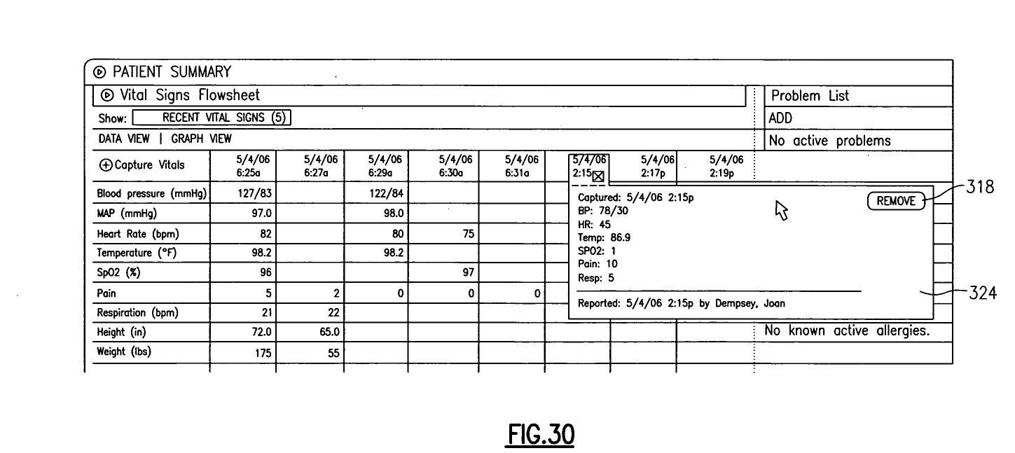

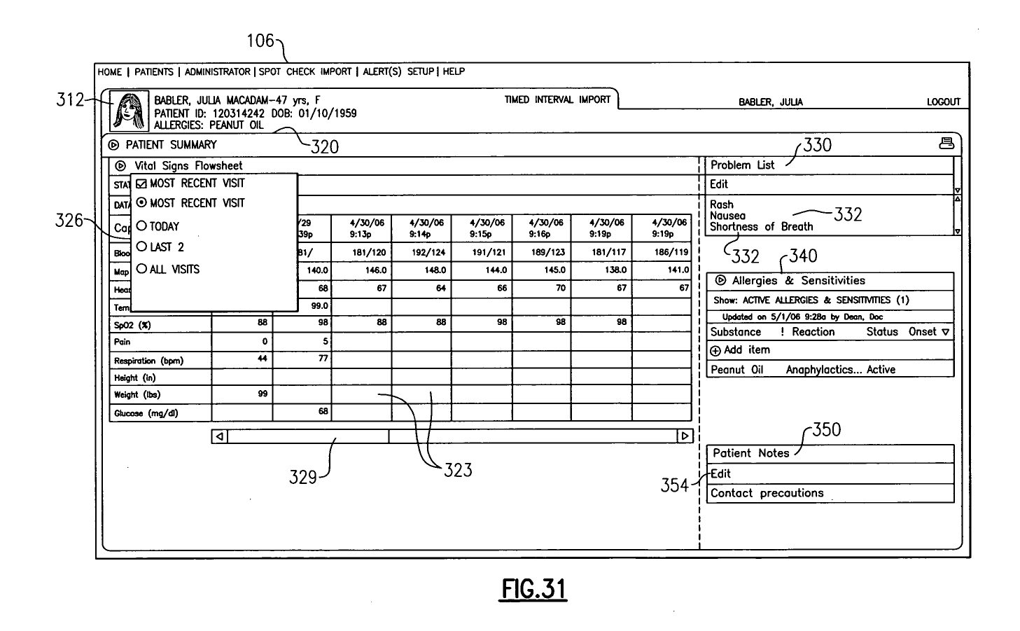

More specifically and referring to FIGS. 29(a)-29(e), a sample allergies and sensitivities window 340 is shown in FIG. 29(a). The window 340 includes each of the foregoing items in the table field 343 including a list of substances and their effect on the patient. Included in this listing is whether the allergy is active or inactive, and a date of onset (if known). Available choices can be toggled between after clicking on the text 345, FIG. 29(a), next to “Show” in the upper portion of the window 340 and referring to a list of available choices 347 (active, removed, all and inactive allergies and sensitivities), as shown in FIG. 29(b). Clicking upon any listed allergen causes the simultaneous display of an allergies reveal window 349, FIG. 29(c). The allergy reveal window 349 displays the status and severity of the allergy and when the allergy was documented. Comments can also be provided in this window 349, if any have previously been added. The reveal window 349 further includes a series of action or navigation buttons 353 used to selectively edit, inactivate and/or remove the entry.

Referring back to the exemplary patient summary display screen 310 and clicking onto the add item button 344 in the allergies and sensitivities window 340 opens an allergies document window 355, examples of which are shown in FIGS. 29(d) and 29(e). Various data fields are included in this window 355 including a scrollable allergy reaction list 351, FIG. 29(e), as well as text and date fields to create an allergy listing or record.

The patient notes window 350 located in the lower right hand corner of the patient summary display screen 310 provides a means for the user to add or review other notes relating to an identified patient. According to this example, the notes concerning this patient indicate that the patient is hypertensive and requests that the attending nurse be notified if the systolic blood pressure is at 160 or greater. By clicking onto an edit icon 354, FIG. 26, the notes can be changed by a user at any time as needed.

As previously noted, vital signs data from other devices, such as other vital sign collecting devices that have already collected readings for a particular patient can be selectively imported into the herein described workstation 10. According to a first aspect, the diagnostic workstation 10 can import vital signs data from a remote stand alone vital signs device 41, such as a Spot Ultra or other monitoring device, as shown in FIG. 6.

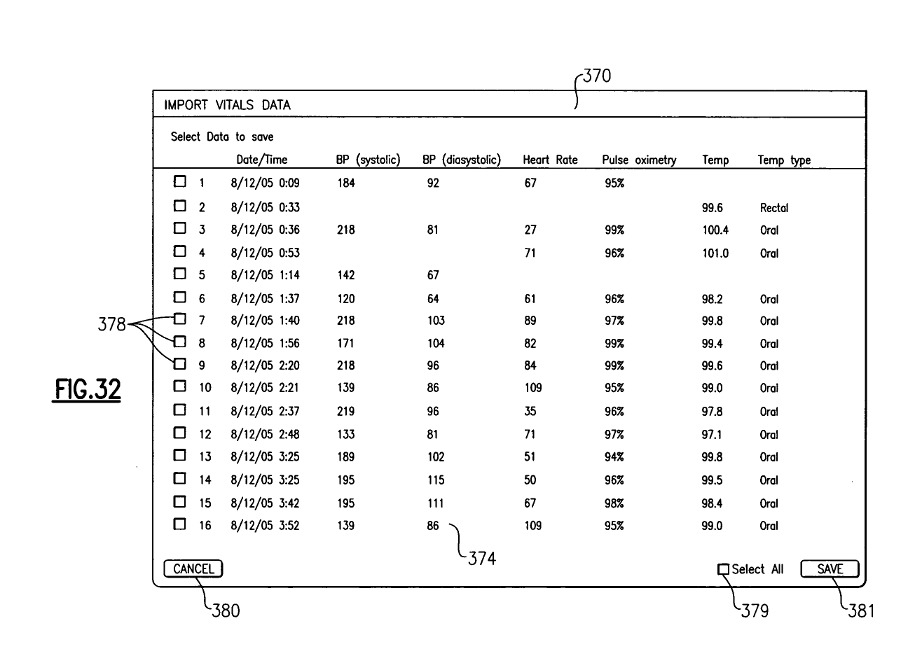

Referring to FIG. 32-34, and by selecting IMPORT DATA from the global header 106 provided at the top of the patient summary or other display screen 310, FIG. 26, for an identified patient, an import vitals data window 370 is displayed. Referring to FIG. 32, the import vitals data window 370 includes an itemized tabular listing 374 of readings taken by the device, including a date/time stamp for each reading, the parameter values (in this case, blood pressure (systolic), blood pressure (diastolic), heart rate, pulse oximetry, temperature and site of temperature measurement (oral, axillary, rectal)).

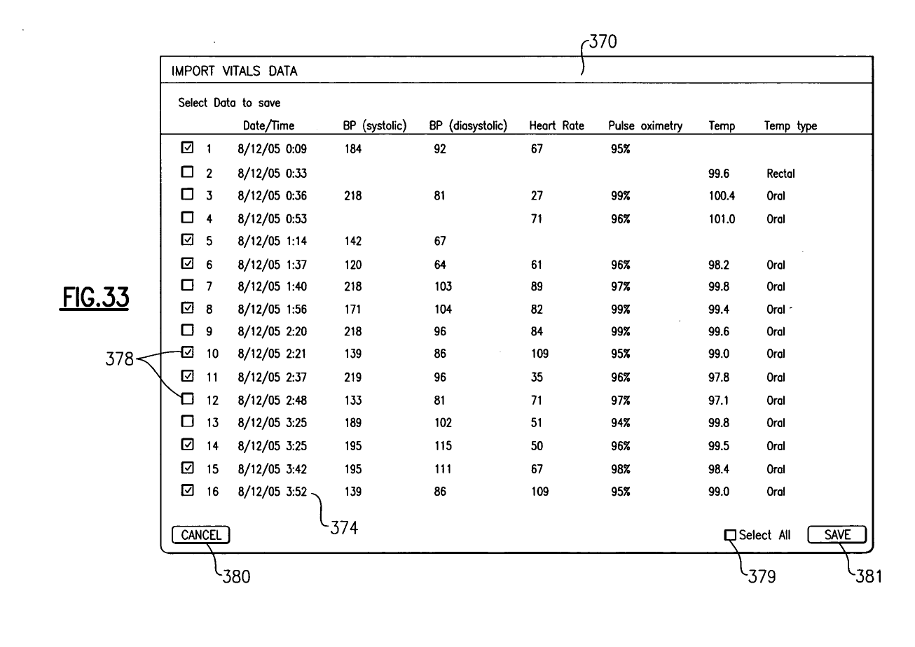

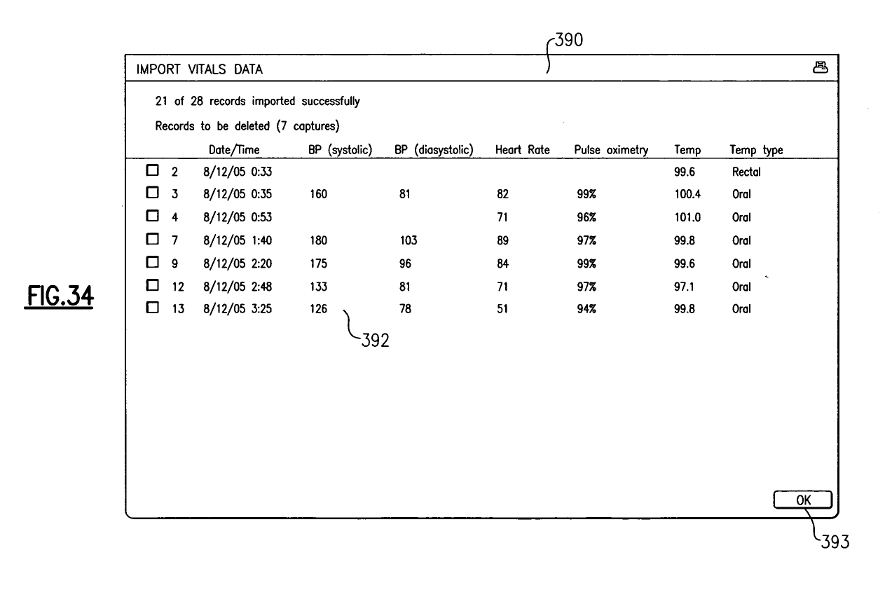

Referring to FIGS. 32 and 33, specific readings can be accepted by the user for import through a series of check boxes 378. A scroll bar (not shown) enables the user to view and make elections on all readings that can be imported from the remote device. A Select All option box 379 is provided in the bottom of the window 370, as well as a cancel button 380 and a save button 381. Clicking onto the cancel button 380 cancels the import data function and restores the clinical summary display screen 310, FIG. 26. Clicking onto the save button 381 produces a display window 390 as shown in FIG. 34. In this display window 390, acknowledgement is made of each of the records imported into the diagnostic workstation 10 and a listing 392 is provided of those readings that were not selected from the remote device. This listing 392 provides the user with an additional opportunity to consider importing or printing the readings prior to deletion, wherein an ok or confirmation button 393 is provided at the bottom of the display window 390.

Users will upload the Spot Ultra Lxi device when using the barcode scanner to identify themselves and patients. These readings can be uploaded to the workstation 10 without patient context. In this embodiment, readings are automatically uploaded. Those readings that cannot be reconciled with patients will be identified. For example, if no patient identifier is provided in that the patient identification wristband was not scanned, or in the instance a patient identification number is incorrect. These specific readings would be displayed to the user, who can print and enter the readings later, if sufficient information is obtained.

PARTS LIST FOR FIGS. 1-36

| | |
|---|---|
| 10 | medical diagnostic workstation |
| 14 | wheeled chassis |
| 18 | computing device |
| 20 | vertically extending post member |
| 22 | base portion |
| 24 | upper portion |
| 25 | legs |
| 26 | vital signs collecting device |
| 27 | casters |
| 28 | horizontal work surface |
| 29 | display, collecting device |
| 30 | display |
| 33 | actuable buttons |
| 34 | keyboard |
| 36 | tray drawer |
| 37 | bracket |
| 38 | mouse |
| 39 | attachment plate |
| 41 | stand-alone device |
| 42 | power source |
| 43 | vital signs device |
| 50 | scanner |
| 54 | retaining cradle |
| 60 | flexible goose neck |
| 64 | ECG electrode assembly |
| 72 | electrodes/leadwires |
| 78 | receptacle |
| 80 | handle |
| 84 | pulse oximeter sensor |
| 100 | log-in display screen |
| 102 | data entry fields |
| 104 | user's display screen |
| 106 | global header |
| 108 | Reminders window |
| 112 | Messages window |
| 116 | List Patients window |
| 117 | location selection window |
| 118 | print icons |
| 119 | ok button |
| 120 | list of patients |
| 121 | date/time stamp |
| 122 | location |
| 123 | vertical scroll bar |
| 124 | search locator |
| 125 | locations header |
| 126 | go button |
| 127 | patients search window |
| 128 | cancel button |
| 129 | search data fields |
| 130 | patient reveal window |
| 131 | clear button |
| 132 | reminders button |
| 133 | view patient summary button |
| 134 | set reminder window |
| 135 | arrows |
| 136 | field, date expiration |
| 137 | action or navigation buttons |
| 140 | text field |
| 150 | reminders reveal window |
| 156 | edit reminders button |
| 160 | message listing |
| 162 | new messages window |
| 163 | address book icon |
| 164 | action buttons |
| 165 | “To” data entry field |
| 166 | send button |
| 167 | subject field |
| 168 | message text field |
| 169 | cancel button |
| 170 | view message window |
| 171 | high priority field |
| 174 | buttons |
| 175 | asterisks |
| 180 | capture vitals button |
| 182 | patient summary button |
| 184 | set alert button |
| 190 | capture vitals display screen |
| 192 | blood pressure data entry window |
| 193 | start button |
| 194 | heart rate data entry window |
| 195 | stop button |
| 196 | temperature data entry window |
| 198 | pulse oximetry data entry window |
| 200 | pain scale entry window |
| 204 | respiration rate data entry window |
| 206 | height data entry window |
| 208 | weight data entry window |
| 210 | glucose data entry window |
| 212 | qualifier data entry windows - blood pressure |
| 214 | qualifier data entry windows - heart rate |
| 216 | qualifier data entry windows - temperature |
| 218 | qualifier data entry windows - pulse oximetry |
| 220 | qualifier data entry windows - respiration |
| 221 | manual entry button |
| 222 | qualifier data entry windows - height |
| 224 | qualifier data entry windows - weight |
| 228 | previous blood pressure reading |
| 230 | previous heart rate reading |
| 232 | previous temperature reading |
| 234 | previous pulse oximetry reading |
| 236 | previous pain index reading |
| 238 | previous respiration rate |
| 240 | previous height reading |
| 242 | previous weight reading |
| 244 | previous glucose reading |
| 248 | patient notes window |
| 252 | time entry field |
| 256 | save button |
| 260 | cancel button |
| 264 | graphical icon |
| 266 | highlighted box |
| 270 | highlighted box |
| 274 | highlighted box |
| 278 | highlighted box |
| 290 | confirmation of vitals capture window |
| 294 | cancel button |
| 298 | modify button |
| 300A | set notifications display screen |
| 300B | set notifications display screen |
| 302 | set notifications values window |
| 304 | parameter selection boxes |
| 306 | selection windows |
| 307 | date field |
| 308 | OK button |
| 309 | cancel button |
| 310 | patient summary display screen |
| 311 | scrollable drop-down box |
| 312 | image - patient |
| 318 | remove button |
| 320 | clinical summary window |
| 321 | data format window |
| 323 | tabular flowsheet format - data readings |
| 324 | patients summary reveal window |
| 325 | graphical display - data readings |
| 326 | data display window |
| 327 | graphical selection boxes |
| 328 | print icon |
| 329 | scroll bar |
| 330 | problems list window |
| 332 | text field |
| 340 | allergies and sensitivities window |
| 343 | table field |
| 344 | Add Item icon |
| 346 | review fill-in box |
| 347 | list - choices |
| 349 | allergies reveal window |
| 350 | patient notes window |
| 351 | allergy reaction list, scrollable |
| 353 | action buttons |
| 354 | edit icon |
| 355 | allergies document window |
| 370 | import vital data window |
| 374 | listing of readings |
| 378 | import data reading selection boxes. |
| 379 | select all option box |
| 380 | cancel button |
| 381 | save button |
| 390 | import vital data window |
| 392 | listing of non-imported readings |
| 393 | OK or confirmation button |

What is claimed is:

1. An electronic data management system, said system comprising:
at least one computing device;
at least one display;
at least one input device; and
at least one vital signs collecting device, wherein said each of said at least one computing device and said at least one display are interconnected to one another in a diagnostic workstation and said at least one vital signs collecting device is at least one of directly integrated into said workstation or is configured for connection therewith, but is not a structural part of said workstation, said at least one computing device being further programmed to receive and manage vital signs readings relating to at least one patient and in which said system includes a plurality of selectively accessible display screens for creating an electronic patient record and managing data relating to the at least one patient.

2. A system as recited in claim 1, wherein said diagnostic workstation can selectively import vital signs data from at least one other vital signs collecting device that is not a structural part of said workstation.

3. A system as recited in claim 2, wherein said at least one other vital signs collecting device is wirelessly connected to said workstation to permit importation of data.

4. A system as recited in claim 1, wherein said computing device is programmed to selectively receive and manage vital signs readings from a plurality of patients.

5. A system as recited in claim 1, wherein said workstation is interconnected to a network and in which data received by said workstation can selectively be uploaded to said network.

6. A system as recited in claim 1, wherein said at least one input device includes at least one of a keyboard, a trackball, a scanning device, a virtual display and a mouse.

7. A system as recited in claim 1, wherein one of said selectively accessible display screens are a user's display screen including a list of patients being handled by said workstation.

8. A system as recited in claim 7, wherein said list of patients of said user's display screen can selectively be arranged selectively by at least one of the name of the listed patient and by location of the patient in a care facility.

9. A system as recited in claim 7, wherein said user's display screen includes the most current vital signs readings taken of each said listed patient.

10. A system as recited in claim 9, including identifiers to indicate vital sign readings that are outside a range of predetermined limits.

11. A system as recited in claim 1, wherein one of said selectively accessible display screens is a vitals capture display screen of a selected patient, said vitals capture display screen including a plurality of data fields for entry and display of vital signs data from at least one of said at least one vital signs collecting device, at least one separate device, and manually entered data.

12. A system as recited in claim 1, wherein one of said selectively accessible display screens is a patient summary display screen listing one of a tabular and a graphical summary of a selected patient's readings.

13. A system as recited in claim 12, wherein the intervals between readings displayed on said patient summary display screen can be selectively adjusted by a user.

14. A system as recited in claim 12, wherein a user can selectively adjust which parameters being measured are to be graphically displayed by said system.

15. A system as recited in claim 12, wherein readings outside of a predetermined range are highlighted on said display.

16. A system as recited in claim 11, wherein said vitals capture display screen includes qualifier data that is selected by a user to indicate various conditions relating to a vital sign reading being measured.

17. A system as recited in claim 16, wherein said qualifier data includes at least one of the method of measurement, the position of a patient during a measurement procedure, and the position of a measuring sensor on a patient.

18. A system as recited in claim 16, wherein said vital capture display screen includes a pain measurement field, said pain measurement field including at least one of an indicator to indicate the degree of pain the patient is in and actions taken and entered by a user to relieve pain.

19. A system as recited in claim 11, wherein said at least one vital signs collecting device includes a blood pressure module, wherein said vitals capture display screen includes a user interface used to selectively activate said blood pressure module.

20. A system as recited in claim 11, wherein said vital signs capture display screen includes indicators to indicate when at least one of an equipment and other failure or error has occurred in taking a vital signs reading.

21. A system as recited in claim 11, wherein said vital signs capture display screen includes indicators when any vital signs reading is not within a predetermined range.

22. A system as recited in claim 11, wherein said vital signs capture display screen includes a user interface to selectively permit both manual and automated capture of vital signs data.

23. A system as recited in claim 21, wherein said workstation is configured to automatically transmit out of range readings to at least one of a nurse's station and at least one other networked workstation or computing device.

24. A system as recited in claim 11, wherein said vital signs display screen permits a user to selectively enter vital sign readings and to selectively sign off on readings that are taken, wherein signed off readings are stored into a database created by said computing device.

25. A system as recited in claim 11, wherein said vital signs capture screen is configured to permit a user to selectively highlight readings taken that look unusual but are still within an acceptable range of values.

26. A system as recited in claim 1, further including a scanning device used to identify at least one of an authorized user and patient.

27. A system as recited in claim 7, wherein a patient can be selected from said user's display screen using said at least one input device, the selection of said patient selectively accessing a patient reveal window, said patient reveal window having additional information in addition to that being displayed.

28. A system as recited in claim 27, wherein said patients reveal window includes demographic and other patient related information not provided on said patients list.

29. A system as recited in claim 27, wherein said patients reveal window further includes a plurality of navigation buttons accessible through said at least one input device to permit selective movement between various display screens of said system.

30. A system as recited in claim 29, wherein said navigation buttons include a capture vitals button, the selective actuation of which by said at least one input device toggles a capture vitals display screen for a revealed patient.

31. A system as recited in claim 29, wherein said navigation buttons include a set reminders button, the selective actuation of which by said at least one input device permits a reminder to be set for a revealed patient.

32. A system as recited in claim 29, wherein said navigation buttons include a patient summary button, the selective actuation of which by said at least one input device toggles a patient summary display screen for a revealed patient.

33. A system as recited in claim 29, wherein said navigation buttons include a set alert button, the selective actuation of which by said at least one input device permits the setting of at least one alert.

34. A system as recited in claim 31, wherein actuation of said set reminder button causes a reminder set window to be displayed for said patient, said reminder set window including at least one field in which information can be entered using said at least one input device.

35. A system as recited in claim 34, wherein a plurality of reminders can be set using a plurality of set reminder windows, and in which each said reminder can be separately saved and deleted.

36. A system as recited in claim 35, wherein each said reminder is defined by a default expiration time, said reminder set window including means for selectively setting the expiration time of at least one reminder to a different time than that of the default.

37. A system as recited in claim 34, wherein all set reminders are displayed in a reminders list.

38. A system as recited in claim 37, wherein said reminders list is selectively accessible by said at least one input device, wherein selection of a reminder by said at least one input device causes the simultaneous display of a reminders reveal window, said reminders reveal window including additional patient related information in addition to that included in the set reminder window.

39. A system as recited in claim 38, wherein said reminders reveal window further includes a plurality of navigation buttons accessible through said at least one input device for selectively accessing other display screens of said system.

40. A system as recited in claim 39, wherein said navigation buttons include a patients summary button, the selective actuation of which causes the toggling of a patients summary display screen for the identified patient.

41. A system as recited in claim 39, wherein said navigation buttons include an edit reminders button, the selective actuation of which using said at least one input device causes the toggling of a set reminders window for the reminder.

42. A system as recited in claim 11, wherein said workstation is configured to receive vital signs readings automatically from said at least one device for entry on said capture vitals display screen or by manual entry thereupon.

43. A system as recited in claim 11, wherein each of the data fields in said vital signs capture display screen includes the previous measurement adjacent thereto.

44. A system as recited in claim 43, wherein said previous measurement includes a time and date stamp.

45. A system as recited in claim 16, wherein said data fields in said capture vitals display screen include fields for entering body temperature, blood pressure, heart rate, pulse oximetry, respiration, weight, height, and glucose, at least one of said data fields including at least one qualifier that is selectively entered.

46. A system as recited in claim 45, wherein the data captured and displayed on said capture vitals display screen can selectively be saved or canceled by said user.

47. A system as recited in claim 11, wherein data saved by said user from said vitals capture display screen is added to a database of said workstation, including a patients summary display screen.

48. A system as recited in claim 1, wherein data imported from said at least one of said at least one vital signs collecting device is selectively saved by a user for storage in a database of said workstation for selective display thereof.

49. A system as recited in claim 46, wherein the contents of a vital capture display screen cannot be saved unless certain mandatory fields have first been entered by a user.

50. A system as recited in claim 33, wherein actuation of said set alert button permits alerts to be set for at least one of locations of patients and for individual patients.

51. A system as recited in claim 50, wherein alerts can be set for individual physiological parameters by setting a predetermined range of permissible parameter values.

52. A system as recited in claim 32, wherein said patient summary display screen includes at least one of a tabular and a graphical display of vital sign readings of an identified patient over a predetermined time interval.

53. A system as recited in claim 52, wherein the predetermined time interval can be selectively adjusted.

54. A system as recited in claim 32, wherein said patients summary display screen further includes a problem list.

55. A system as recited in claim 32, wherein said patients summary display screen further includes a listing of allergies for the identified patient.

56. A system as recited in claim 32, wherein said patients summary display screen further includes a list of patient notes associated with said identified patient.

57. A system as recited in claim 32, wherein the vital sign readings displayed in said patient summary display screen include those readings displayed and saved in the capture vitals display screen.

58. A system as recited in claim 54, wherein said problems list permits selective listing of a patient's complaints and conditions, said problems list being selectively editable by a user within said patients summary display screen.

59. A system as recited in claim 55, wherein said listing of allergies is editable within said patients summary display screen.

60. A system as recited in claim 59, including an allergies reveal window that is selectively accessible using said at least one input device, said allergies reveal window including means for editing a listed allergy.

61. A system as recited in claim 60, wherein said allergies reveal window includes a plurality of buttons, the actuation of which permit editing and/or removal of said listed allergy.

62. A system as recited in claim 59, wherein active and inactive allergies can be listed in said listing of allergies, each of said active and inactive allergies listing being accessible from said patients summary screen.

63. A system as recited in claim 52, wherein each of said vital signs readings displayed in said patients summary display screen can be accessed by said at least one input device, wherein said accessing causes the simultaneous display of a patients summary reveal window, said patients summary reveal window including additional information concerning said patient.

64. A system as recited in claim 63, wherein out of range readings are highlighted in said reveal window.

65. A system as recited in claim 63, wherein said patients summary reveal window includes at least one button selectively accessible by said at least one input device to permit removal of said vital signs record.

66. A system as recited in claim 7, wherein said patient display screen includes a reminders window listing at least one reminder relating to at least one listed patient.

67. A system as recited in claim 7, wherein said patients display screen further includes a messages window listing messages sent to users of said workstation.

68. A system as recited in claim 67, wherein said workstation is connectable to a network such that incoming and outgoing network-based messages can be stored in said messages window.

69. A system as recited in claim 37, wherein said reminders can be prioritized.

70. A system as recited in claim 67, wherein messages in said messages box can be prioritized.

71. A system as recited in claim 70, wherein a user cannot log off from said workstation until all high priority messages have been answered.

72. A system as recited in claim 24, wherein a user can print data that is not entered, allowing the user the option to manually enter this data at a later time.

73. A system as recited in claim 51, wherein alerts are prioritized based on potential severity.

74. A system as recited in claim 73, wherein the severity of an alert is sent with said alert to a nurse's station to permit alerts to be prioritized remotely.

75. A system as recited in claim 32, wherein an image of the patient is provided on said patient summary display screen.

76. A system as recited in claim 75, wherein said image is retrieved from a database in said computing device.

77. A system as recited in claim 75, wherein said workstation includes an imaging device for capturing said image.

78. A system as recited in claim 26, wherein said scanning device is used to scan a patient's identification wristband prior to capture of vitals data.

79. A system as recited in claim 5, wherein data is transmitted over the network using an encryption protocol.

80. A system as recited in claim 5, wherein said network is a hospital network.

81. A system as recited in claim 5, wherein data is transmitted for storage to a networked information system using an HL7 protocol.

82. A system as recited in claim 1, wherein said computing device includes at least one of a laptop computer, a tablet PC and a portable data terminal (PDT).

83. A system as recited in claim 45, wherein said qualifier data fields in said capture vitals display screen are customizable, depending on the location of the workstation.

84. A system as recited in claim 20, wherein said computing device stores and tracks the occurrence of equipment failures.

85. A system as recited in claim 50, wherein at least some alerts are highlighted on said display.

86. A system as recited in claim 50, wherein set alerts can expire after a predetermined time interval.

87. A system as recited in claim 86, wherein said time interval is selectable.

88. A system as recited in claim 1, wherein said computing device can store trend data relating to readings relating to at least one patient.

89. A system as recited in claim 2, wherein a list of patients can be downloaded from said workstation to a remote connected device.

90. A system as recited in claim 24, wherein said capture vitals display screen includes a save button permitting sign off of vitals captured by said workstation.

91. A system as recited in claim 5, including at least one of a PDT and a Tablet PC introduced between said at least one remote vital signs collecting device and said network.

92. An electronic patient data management system, said system comprising:
a computing device;
at least one display; and
at least one vital signs collecting device, each of said computing device, said at least one display and said at least vital signs collecting device being interconnected to one another in a diagnostic workstation, wherein data relating to at least one patient is captured and managed using a plurality of selectively accessible electronic display screens.

93. A system as recited in claim 92, including at least one input device interconnected to said workstation.

94. A system as recited in claim 93, wherein said at least one input device includes at least one of a mouse, a trackball, a keyboard, a scanner device, and a virtual display.

95. A system as recited in claim 93, wherein data from at least one other vital signs collecting device and relating to at least one patient that is not structurally part of said workstation can be selectively imported to said workstation.

96. A system as recited in claim 95, wherein portions of data from said at least one other vital signs collecting device can be selectively imported to said workstation.

97. A system as recited in claim 92, wherein said selective accessible display screens include a patients display screen including a list of patients being handled by said workstation.

98. A system as recited in claim 97, wherein said patients display screen includes the most current vital signs readings of each listed patient.

99. A system as recited in claim 98, wherein values of vital sign readings that exceed a range of predetermined values are highlighted.

100. A system as recited in claim 97, including an imbedded reveal window accessible by highlighting a patient on said list using said at least one input device.

101. A system as recited in claim 100, wherein said imbedded reveal window includes additional information relating to the selected patient.

102. A system as recited in claim 100, wherein said reveal window further includes a plurality of navigation buttons to enable a user to navigate between said selectively accessible display screens.

103. A system as recited in claim 102, wherein one of said navigation buttons is a create vitals button, the actuation of which toggles a capture vitals capture display screen.

104. A system as recited in claim 102, wherein one of said navigation buttons is a create reminders button, the actuation of which toggles a create reminders display screen.

105. A system as recited in claim 102, wherein one of said navigation buttons is a create alerts buttons, the actuation of which toggles a create alerts display screen.

106. A system as recited in claim 93, wherein said display screens are selectively accessed using imbedded reveal windows in each said display screen format, said reveal windows each being accessible using said at least one input device and in which said reveal windows further include navigation controls to access various display screens.

107. A system as recited in claim 106, wherein said display screens include a user's display screen, said user's display screen further including an imbedded patient reveal window.

108. A system as recited in claim 97, wherein said list of patients of said user's display screen can selectively be arranged selectively by at least one of the name of the listed patient and by location of the patient in a care facility.

109. A system as recited in claim 107, wherein said patients reveal window includes demographic and other patient related information not provided on said patients list.

110. A system as recited in claim 107, wherein said patients reveal window further includes a plurality of navigation buttons accessible through said at least one input device to permit selective movement between various display screens of said system.

111. A system as recited in claim 110, wherein said navigation buttons include a capture vitals button, the selective actuation of which by said at least one input device toggles a capture vitals display screen for a revealed patient.

112. A system as recited in claim 110, wherein said navigation buttons include a set reminders button, the selective actuation of which by said at least one input device permits a reminder to be set for a revealed patient.

113. A system as recited in claim 110, wherein said navigation buttons include a patient summary button, the selective actuation of which by said at least one input device toggles a patient summary display screen for a revealed patient.

114. A system as recited in claim 110, wherein said navigation buttons include a set alert button, the selective actuation of which by said at least one input device permits the setting of at least one alert.

115. A system as recited in claim 112, wherein actuation of said set reminder button causes a reminder set window to be displayed for said patient, said reminder set window including at least one field in which information can be entered using said at least one input device.

116. A system as recited in claim 115, wherein a plurality of reminders can be set using a plurality of set reminder windows, and in which each said reminder can be separately saved and deleted.

117. A system as recited in claim 116, wherein each said reminder is defined by a default expiration time, said reminder set window including means for selectively setting the expiration time of at least one reminder to a different time than that of the default.

118. A system as recited in claim 116, wherein all set reminders are displayed in a reminders list.

119. A system as recited in claim 118, wherein said reminders list is selectively accessible by said at least one input device, wherein selection of a reminder by said at least one input device causes the simultaneous display of a reminders reveal window, said reminders reveal window including additional patient related information in addition to that included in the set reminder window.

120. A system as recited in claim 119, wherein said reminders reveal window further includes a plurality of navigation buttons accessible through said at least one input device for selectively accessing other display screens of said system.

121. A system as recited in claim 120, wherein said navigation buttons include a patients summary button, the selective actuation of which causes the toggling of a patients summary display screen for the identified patient.

122. A system as recited in claim 120, wherein said navigation buttons include an edit reminders button, the selective actuation of which using said at least one input device causes the toggling of a set reminders window for the reminder.

123. A system as recited in claim 97, wherein said user's display screen includes a reminders window listing at least one reminder relating to at least one listed patient.

124. A system as recited in claim 97, wherein said user's display screen further includes a messages window listing messages sent to users of said workstation.

125. A system as recited in claim 124, wherein said workstation is connectable to a network such that incoming and outgoing network-based messages can be stored in said messages window.

126. A system as recited in claim 123, wherein said reminders can be prioritized.

127. A system as recited in claim 124, wherein messages in said messages window can be prioritized.

128. A system as recited in claim 127, wherein a user cannot log off from said workstation until all high priority messages have been answered.

129. A system as recited in claim 92, wherein one of said selectively accessible display screens is a vitals capture display screen of a selected patient, said vitals capture display screen including a plurality of data fields for entry and display of vital signs data from at least one of said at least one vital signs collecting device, at least one separate device, and manually entered data.

130. A system as recited in claim 129, wherein said vitals capture display screen includes qualifier data that is selected by a user to indicate various conditions relating to a vital sign reading being measured.

131. A system as recited in claim 130, wherein said qualifier data includes at least one of the method of measurement, the position of a patient during a measurement procedure, and the position of a measuring sensor on a patient.

132. A system as recited in claim 130, wherein said vital capture display screen includes a pain measurement field, said pain measurement field including an indicator to indicate the degree of pain the patient is in and actions taken and entered by said user to relieve pain.

133. A system as recited in claim 129, wherein said at least one vital signs collecting device includes a blood pressure module, wherein said vitals capture display screen includes a user interface used to selectively activate said blood pressure module.

134. A system as recited in claim 129, wherein said vital signs capture display screen includes indicators to indicate when at least one of an equipment and other failure or error has occurred in taking a vital signs reading.

135. A system as recited in claim 129, wherein said vital signs capture display screen highlights or otherwise indicates any vital signs reading that is not within a predetermined range.

136. A system as recited in claim 129, wherein said vital signs capture display screen includes a user interface to selectively permit both manual and automated capture of vital signs data.

137. A system as recited in claim 136, wherein said workstation is configured to automatically transmit out of range readings to at least one of a nurse's station, a networked workstation and a networked PC.

138. A system as recited in claim 129, wherein said vital signs display screen permits a user to selectively enter vital sign readings and to selectively sign off on readings that are taken, wherein signed off readings are stored into a database created by said computing device.

139. A system as recited in claim 129, wherein said vital signs capture screen is configured to permit a user to selectively highlight readings taken that look unusual but are still within an acceptable range of values.

140. A system as recited in claim 129, wherein said workstation is configured to receive vital signs readings automatically from said at least one device for entry on said capture vitals display screen or by manual entry thereupon.

141. A system as recited in claim 129, wherein each of the data fields in said vital signs capture display screen includes the previous measurement adjacent thereto.

142. A system as recited in claim 141, wherein said previous measurement includes a time and date stamp.

143. A system as recited in claim 130, wherein said data fields in said capture vitals display screen include fields for entering body temperature, blood pressure, heart rate, pulse oximetry, respiration, weight, height, and glucose, at least one of said data fields including at least one qualifier that is selectively entered.

144. A system as recited in claim 130, wherein the data captured and displayed on said capture vitals display screen can selectively be saved or canceled by said user.

145. A system as recited in claim 144, wherein data saved by said user from said vitals capture display screen is added to a database of said workstation, including a patients summary display screen.

146. A system as recited in claim 144, wherein the contents of a vital capture display screen cannot be saved unless certain mandatory fields have first been entered by a user.

147. A system as recited in claim 146, wherein said mandatory fields includes at least one predetermined qualifier.

148. A system as recited in claim 144, wherein readings that are out of a predetermined range are highlighted on said vitals capture display screen.

149. A system as recited in claim 92, wherein one of said selectively accessible display screens is a patient summary display screen listing one of a tabular and a graphical summary of a selected patient's readings.

150. A system as recited in claim 149, wherein the intervals between readings displayed on said patient summary display screen can be selectively adjusted by a user.

151. A system as recited in claim 149, wherein a user can selectively adjust which parameters being measured are to be graphically displayed by said system.

152. A system as recited in claim 149, wherein said patient summary display screen includes vital sign readings of an identified patient over a predetermined time interval.

153. A system as recited in claim 152, wherein the predetermined time interval can be selectively adjusted.

154. A system as recited in claim 149, wherein said patients summary display screen further includes a problem list.

155. A system as recited in claim 149, wherein said patients summary display screen further includes a listing of allergies for the identified patient.

156. A system as recited in claim 149, wherein said patients summary display screen further includes a list of patient notes associated with said identified patient.

157. A system as recited in claim 149, wherein the vital sign readings displayed in said patient summary display screen include those readings displayed and saved in the capture vitals display screen.

158. A system as recited in claim 154, wherein said problems list permits selective listing of a patient's complaints and conditions, said problems list being selectively editable by a user within said patients summary display screen.

159. A system as recited in claim 155, wherein said listing of allergies is editable within said patients summary display screen.

160. A system as recited in claim 159, including an allergies reveal window that is selectively accessible using said at least one input device, said allergies reveal window including means for editing a listed allergy.

161. A system as recited in claim 160, wherein said allergies reveal window includes a plurality of buttons, the actuation of which permit editing and/or removal of said listed allergy.

162. A system as recited in claim 155, wherein active and inactive allergies can be listed in said listing of allergies, each of said active and inactive allergies listing being accessible from said patients summary screen.

163. A system as recited in claim 149, wherein each of said vital signs readings displayed in said patients summary display screen can be accessed by said at least one input device, wherein said accessing causes the simultaneous display of a patients summary reveal window, said patients summary reveal window including additional information concerning said patient.

164. A system as recited in claim 163, wherein out of range readings are highlighted in said reveal window.

165. A system as recited in claim 163, wherein said patients summary reveal window includes at least one button selectively accessible by said at least one input device to permit removal of said vital signs record.

166. A system as recited in claim 114, wherein actuation of said set alert button permits alerts to be set for at least one of locations of patients and for individual patients.

167. A system as recited in claim 166, wherein alerts can be individually set for specific physiological parameters by creating a predetermined range of permissible parameter values.

168. A system as recited in claim 138, in which said workstation is connectable to a printer, wherein a user can print data that is not able to be stored by said workstation, allowing the user the option to manually enter this data at a later time.

169. A system as recited in claim 167, wherein alerts are prioritized based on potential severity.

170. A system as recited in claim 169, wherein the severity of an alert is sent with said alert to at least one networked location to permit an alert to be prioritized remotely.

171. A system as recited in claim 169, wherein said at least one networked location is a nurse's station.

172. A system as recited in claim 92, further including a scanning device used to identify one of an authorized user and patient.

173. A system as recited in claim 149, wherein an image of the patient is provided on said patient summary display screen.

174. A system as recited in claim 172, wherein said scanning device is used to scan a patient's identification wristband prior to capture of vitals data.

175. A system as recited in claim 92, wherein said workstation is interconnected to a network and in which data received by said workstation can selectively be uploaded to said network.

176. A system as recited in claim 175, wherein said network is a hospital network.

177. A system as recited in claim 175, wherein data is transmitted over the network using an encryption protocol.

178. A system as recited in claim 175, wherein data is transmitted for storage to a networked information system using an HL7 protocol.

179. A system as recited in claim 92, wherein said computing device includes at least one of a laptop computer, a tablet PC and a portable data terminal (PDT).

180. A system as recited in claim 129, wherein said qualifier data fields in said capture vitals display screen are customizable, depending on the location of the workstation.

181. A system as recited in claim 134, wherein said computing device stores and tracks the occurrence of equipment failures.

182. A system as recited in claim 169, wherein at least some alerts are highlighted.

183. A system as recited in claim 114, wherein set alerts can expire after a predetermined time interval.

184. A system as recited in claim 183, wherein said time interval is selectable.

185. A system as recited in claim 92, wherein said computing device stores trend data relating to readings relating to at least one patient.

186. A system as recited in claim 95, wherein a list of patients can be downloaded to a remote connected device.
